(19) United States
(12) Patent Application Publication
(10) Pub. No.: US 2022/0314078 A1
(43) Pub. Date: Oct. 6, 2022

(54) VIRTUAL ENVIRONMENT WORKOUT CONTROLS

(71) Applicant: ICON HEALTH & FITNESS, INC. n/k/a iFit, Inc., Logan, UT (US)

(72) Inventors: Eric Watterson, Logan, UT (US); Nick Watterson, Logan, UT (US); Joseph A. Torres, JR., Tarzana, CA (US); Michael Hope, Chapin, SC (US)

(21) Appl. No.: 17/712,347

(22) Filed: Apr. 4, 2022

Related U.S. Application Data
(60) Provisional application No. 63/200,903, filed on Apr. 2, 2021.
(60) Provisional application No. 63/259,904, filed on Dec. 28, 2021.

(51) Int. Cl. A63B 24/00; A63B 21/22; A63B 22/02; A63B 71/06

(52) U.S. Cl. CPC A63B 24/0087; A63B 21/225; A63B 22/02; A63B 24/0075; A63B 71/0622; A63B 2024/009; A63B 2024/0068; A63B 2071/0644; A63B 2220/62; A63B 2071/0625

(57) ABSTRACT

In one aspect of the disclosure, a method includes rendering, by a processor, a virtual environment associating, by the processor, exercise machine control signals with the virtual environment, and displaying, by the processor, the virtual environment on a video wall. The method may include receiving, by the processor, a video of a trainer on an exercise machine in front of the video wall displaying the virtual environment, wherein the exercise machine has one or more parameters which are controlled according to the exercise machine control signals associated with the virtual environment, associating, by the processor, the control signals with the video of the trainer in the virtual environment, and publishing the video with the associated control signals for use on a remote exercise machine.

This application claims the benefit of and priority to U.S. Provisional Patent Application No. 63/200,903 filed Apr. 2, 2021 and U.S. Provisional Patent Application No. 63/259,904 filed Dec. 28, 2021 which applications are incorporated herein by reference in their entirety.

### BACKGROUND

Mental health maladies are often treated with therapy, counseling, and/or medication. Mental health maladies may also be reduced with exercise; for example, anxiety, depression, and negative mood may be reduced by exercise and self-esteem and cognitive function may be improved by exercise. Exercise may also alleviate symptoms such as low self-esteem and social withdrawal.

Treatment of mental health maladies during or in connection with exercise may be more effective than treatment by itself. Moreover, treatment of mental health maladies during or in connection with exercise may lack the negative side effects that are sometimes associated with medications.

The subject matter claimed herein is not limited to embodiments that solve any disadvantages or that operate only in environments such as those described above. Rather, this background is only provided to illustrate one example technology area where some embodiments described herein may be practiced.

### SUMMARY

In one aspect of the disclosure, a method to generate a video workout program may include capturing a first video that includes a depiction of a trainer performing a workout; combining the depiction of the trainer in the first video with a second video that moves through an environment to form a combined video in which the trainer appears to move through the environment; and encoding exercise machine control commands into a subtitle stream of the combined video to create the video workout program, execution of the video workout program on a first exercise machine configured to display the combined video and continually control one or more moveable members of the first exercise machine according to the exercise machine control commands.

Another aspect of the disclosure may include any combination of the above-mentioned features and may further include the trainer performing the workout using a second exercise machine, monitoring operating parameters of the second exercise machine during performance of the workout by the trainer; and generating the exercise machine control commands to correspond to the depiction of the workout by the trainer, including generating the exercise machine control commands to cause the first exercise machine to implement at least some of the operating parameters of the second exercise machine during execution of the video workout program on the first exercise machine.

Another aspect of the disclosure may include any combination of the above-mentioned features and may further include the trainer performing the workout using a second exercise machine, the second video that moves through the environment including a rendered video that moves through a virtual environment, monitoring a speed of the second exercise machine during performance of the workout by the trainer; and synchronizing a speed at which the rendered video moves through the virtual environment with the speed of the second exercise machine.

Another aspect of the disclosure may include any combination of the above-mentioned features and may further include the capturing the first video that includes the depiction of the trainer performing the workout including capturing the first video of the trainer performing the workout on a second exercise machine in front of a chroma key screen of a stage or set.

Another aspect of the disclosure may include any combination of the above-mentioned features and may further include displaying the second video in view of a camera that captures the first video of the trainer performing the workout, the combining the depiction of the trainer in the first video with the second video including capturing the first video of both the trainer performing the workout and the second video displayed in the view of the camera.

Another aspect of the disclosure may include any combination of the above-mentioned features and may further include receiving input effective to at least one of: control weather or natural phenomena depicted in the second video or add, delete, move, or resize an object in the environment.

Another aspect of the disclosure may include any combination of the above-mentioned features and may further include the combining the depiction of the trainer in the first video with the second video including combining the depiction of the trainer in the first video with the second video in real-time as the trainer performs the workout, streaming the combined video live to the first exercise machine; reaching a branch point in a path traveled in the second video, the path splitting into multiple branches at the branch point; receiving feedback from a first user of the first exercise machine including a selection by the first user of one of the multiple branches of the path to travel down from the branch point; and causing the second video in real-time to travel down the selected branch from the branch point such that the trainer appears to travel down the selected path from the branch point.

Another aspect of the disclosure may include any combination of the above-mentioned features and may further include reaching a branch point in a path traveled in the second video, the path splitting into a first branch and a second branch at the branch point, the combining the depiction of the trainer in the first video with the second video including combining the depiction of the trainer in the first video with the second video as the second video travels along the first branch to form a first selectable portion of the combined video; and combining the depiction of the trainer in the first video with the second video as the second video travels along the second branch to form a second selectable portion of the combined video.

Another aspect of the disclosure may include any combination of the above-mentioned features and may further include encoding environmental control commands into the subtitle stream of the combined video, the environmental control commands configured to control one or more environmental control devices in a vicinity of the first exercise machine.

Another aspect of the disclosure may include any combination of the above-mentioned features and may further include, or may stand alone by including, a method to alter a virtual background of a user on an exercise machine. The method may include capturing, by a camera, a first image or video of a user of an exercise machine with a chroma key screen as an actual background for the user of the exercise machine; combining a depiction of the user in the first image or video with a second image or video to form a combined image or video with a virtual background in place of the actual background; and displaying the combined image or video to at least one of the user or a viewer.

Another aspect of the disclosure may include any combination of the above-mentioned features and may further include the combined image or video being the combined video, establishing a video conference between the user of the exercise machine and another user of another exercise machine, and the displaying the combined video to the at least one of the user or the viewer including displaying the combined video to the user and the other user.

Another aspect of the disclosure may include any combination of the above-mentioned features and may further include displaying a leaderboard with an entry for the user and another entry for another user, the leaderboard ranking performance indicators of the user and the other user with respect to performance of a workout by the user and the other user, the displaying the combined image or video to the at least one of the user or the viewer including displaying the combined image or video within the entry of the user in the leaderboard.

Another aspect of the disclosure may include any combination of the above-mentioned features and may further include executing, at the exercise machine, a video workout program to enable the user to perform a workout on the exercise machine, including displaying a workout video to the user that depicts an environment, the second image or video depicting the environment; and the combined image or video showing the user in the environment.

Another aspect of the disclosure may include any combination of the above-mentioned features and may further include receiving input from the user effective to interact with the environment; and altering the environment in the workout video or the combined image or video responsive to the input.

Another aspect of the disclosure may include any combination of the above-mentioned features and may further include the user performing a workout on the exercise machine and other users performing the workout on other exercise machines; displaying the combined image or video including displaying the depiction of the user and the virtual background in a first block of a multi-user grid where the virtual background displayed in the first block includes a performance indicator of the user in performing the workout; and displaying the grid with the block for the user and a different block for each of the other users, each block of the other users including a combined image or video of a depiction of the corresponding user and a corresponding virtual background, each corresponding virtual background including a performance indicator of the corresponding user performing the workout.

Another aspect of the disclosure may include any combination of the above-mentioned features and may further include the second image or video including one or more virtual beings and the combined image or video showing the one or more virtual beings chasing the user.

Another aspect of the disclosure may include any combination of the above-mentioned features and may further include, or may stand alone by including, a method to execute a video workout program at an exercise machine to enable a user to perform a workout on the exercise machine. The method may include continually controlling one or more moveable members of the exercise machine according to exercise machine control commands of the video workout program; and displaying a video to the user that depicts an environment, the video including multiple viewpoints of the environment, including: displaying a first viewpoint of the video to the user on a first display located in a first position relative to the user; and displaying a second viewpoint of the video to the user on a second display located in a second position relative to the user, the second position different than the first position.

Another aspect of the disclosure may include any combination of the above-mentioned features and may further include at least one of the first display or the second display being movable relative to the exercise machine.

Another aspect of the disclosure may include any combination of the above-mentioned features and may further include the video being a first video, capturing, by a camera, a second video of the user of the exercise machine with the second viewpoint of the first video on the second display device as a background of the user; and displaying the second video to at least one of the user or a viewer.

Another aspect of the disclosure may include any combination of the above-mentioned features and may further include the second display being located behind the user and the second viewpoint of the video includes one or more virtual beings that appear to be chasing the user.

It is to be understood that both the foregoing summary and the following detailed description are explanatory and are not restrictive of the invention as claimed.

### BRIEF DESCRIPTION OF THE DRAWINGS

Embodiments will be described and explained with additional specificity and detail through the use of the accompanying drawings in which:

FIG. 1 illustrates a flowchart of an example wellness device system;

FIG. 2 illustrates a block diagram of an example exercise machine or immersive mental health device;

FIG. 3 illustrates a block diagram of an example sleep assistance device;

FIG. 4 illustrates a block diagram of an example smart yoga mat system;

FIG. 5 illustrates a block diagram of an example smart blanket;

FIGS. 6A and 6B illustrate perspective views of an example immersive mental health device;

FIGS. 7A-7H illustrate perspective views of various example sleep assistance devices;

FIGS. 8A-8C illustrate various views of an example smart blanket;

FIG. 9 illustrates an example smart yoga mat system;

FIG. 10 illustrates a frame of a video of a video mental health program;

FIG. 11 illustrates a frame of a video of a video program that may include both a workout and a mental health improvement session;

FIG. 12 illustrates a frame of a video of another video mental health program;

FIG. 13 illustrates a flowchart of an example method to influence mental state of a user of a wellness device with a video program;

FIG. 14 illustrates a flowchart of an example method to help a user of a sleep assistance device sleep;

FIG. 15 illustrates a flowchart of an example method to make a person accountable to change behavior or mental state from an initial behavior or mental state to a target behavior or mental state;

FIG. 16 illustrates a flowchart of an example method to improve a mental health of a user of one or more wellness devices; and

FIG. 17 illustrates an example computer system that may be employed in performing or controlling performance of one or more of the methods or actions herein.

FIG. 18 illustrates an example view of an example virtual environment.

FIG. 19 illustrates an example view of an example virtual environment with control signal checkpoints.

FIG. 20 illustrates an example view of trainer running in an example virtual environment.

FIG. 21 illustrates a flowchart of an example method for updating control signals at checkpoints in a virtual environment.

FIG. 22 illustrates a flowchart of an example method for automatically associating control signals with points in a virtual environment.

FIG. 23 illustrates a flowchart of an example method for creating and publishing a video of a trainer in a virtual environment.

FIG. 24 illustrates a flowchart of an example method for controlling movement of a virtual trainer through a virtual environment.

FIG. 25 illustrates a flowchart of an example method for controlling a virtual environment using equipment.

FIG. 26 illustrates an example stage for equipment in accordance with one or more embodiments.

Throughout the drawings, identical reference numbers designate similar, but not necessarily identical, elements.

### DETAILED DESCRIPTION

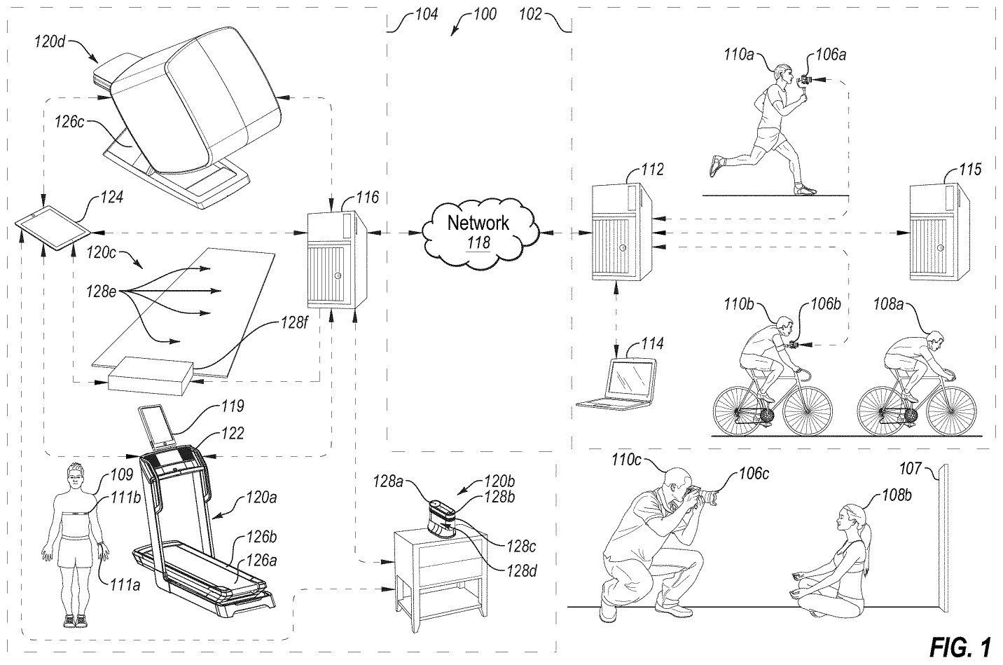

Turning now to the drawings, FIG. 1 illustrates a flowchart of an example wellness device system 100. The wellness device system 100 may include a remote location 102 and a local location 104 connected by a network 118.

In some embodiments, the network 118 may be configured to communicatively couple any two devices in the wellness device system 100 to one another, and/or to other devices. In some embodiments, the network 118 may be any wired or wireless network, or combination of multiple networks, configured to send and receive communications between systems and devices. In some embodiments, the network 118 may include a Personal Area Network (PAN), a Local Area Network (LAN), a Metropolitan Area Network (MAN), a Wide Area Network (WAN), a Storage Area Network (SAN), the Internet, or some combination thereof. In some embodiments, the network 118 may also be coupled to, or may include, portions of a telecommunications network, including telephone lines, for sending data in a variety of different communication protocols, such as a cellular network or a Voice over IP (VoIP) network.

In the remote location 102, the wellness device system 100 may include one or more video cameras 106a, 106b, 106c (hereinafter collectively “video cameras 106” or generically “video camera 106”) that may be employed to capture video for use in a video program as described herein. One or more of the video cameras 106 may include stabilization capabilities to avoid the captured video from being unduly shaky. The video captured by the video cameras may be used in video programs such as video workout programs and/or video mental health programs. The video may be captured by a videographer 110a, 110b, 110c and in some embodiments may include an instructor 108a, 108b performing or directing a workout and/or a mental health improvement session, such as a mindfulness session, a breathing session, a yoga session, a therapy session, or a sleep assistance session. Each instructor 108a, 108b may include a personal trainer, a yoga instructor, a therapist, or other person that provides instructions and/or commentary in the video with respect to the workout or mental health improvement session being performed or directing by the instructor 108a, 108b.

Mindfulness sessions as described herein may include meditation in which a person (e.g., an instructor or user) focuses on being intensely aware of what is being sensed and felt in the moment, without interpretation or judgment. Alternatively or additionally, mindfulness sessions may involve breathing methods, guided imagery, and/or other practices to relax the body and mind and help reduce stress. In a mindfulness session captured on video according to embodiments herein, an instructor may direct users regarding one or more breathing methods, imagery to envision in the users' minds, and/or other practices.

Yoga sessions as described herein may include breath awareness, a warmup involving one or more static or dynamic yoga poses, a series of static or dynamic yoga poses to develop strength and flexibility, pranayama (advanced breathing techniques), meditation, led relaxation, and/or other practices. In a yoga session captured on video according to embodiments herein, an instructor may direct users regarding breath awareness, yoga poses, pranayama, meditation, relaxation, and/or other practices.

Breathing sessions as described herein may include breath awareness, pranayama, or other breathing methods or techniques. In a breathing session captured on video according to embodiments herein, an instructor may direct users regarding breath awareness, pranayama, or other breathing methods or techniques.

Therapy sessions as described herein may include asking users one or more questions, providing users guidance or counseling, or other mentally therapeutic practices. In a therapy session captured on video according to embodiments herein, an instructor may ask users questions, provide users guidance or counseling, and/or direct users with respect to other mentally therapeutic practices.

Sleep assistance sessions as described herein may include one or more aural, visual, olfactory, and/or tactile stimuli that are collectively configured to assist a user to reach and remain in a sleep state and/or to wake from a sleep state. For example, a sleep assistance session may include aural stimuli such as calming music and/or an instructor directing a user in mental relaxation, visual stimuli such as images and/or video of a calming scene, olfactory stimuli such as a scent of lavender, and/or tactile stimuli such as a vibration of a haptic device.

The videos used in video programs herein may be captured and/or generated in any suitable manner. In an embodiment, an instructor may perform or direct a workout or mental health improvement session on location and a videographer may capture video of the instructor as the instructor performs or directs the workout or mental health improvement session on location. For example, the videographer 110b may use the video camera 106b to capture video of the instructor 108a (e.g., a trainer) performing a workout in which the instructor 108a rides a bicycle in a live road bicycle race. In another embodiment, an instructor may perform or direct a workout or mental health improvement session on a set or stage in front of one or more chroma key screens or display panels and the video of the instructor performing or directing the workout or mental health improvement session may be combined with an image or video of a scene or moving through an environment using chroma keying or other suitable technology. For example, the videographer 110c may use the video camera 106c to capture video of the instructor 108b performing or directing a mental health improvement session in front of one or more chroma key screens or display panels 107, hereinafter “backdrop 107”. The video of the instructor 108b may be combined with an image or video, referred to as a background image or video, e.g., by a chroma key process where the backdrop 107 includes one or more chroma key screens or by displaying the background image or video on the backdrop 107 while the video of the instructor 108b is captured where the backdrop 107 includes one or more display panels. The wellness device system 100 may include a remote server 112 that may be configured to combine the video of the instructor with the background image or video, to format video according to one or more formats, or perform other methods or operations described herein.

Background images or videos that may be combined with videos of instructors performing or directing workouts or mental health improvement sessions may include captured images or video, rendered images or video, or a combination of the two. As used herein, a captured image or video refers to an image or video captured by a video camera filming in the real world. A videographer with a video camera may capture video of the real world while the videographer is static or in motion (e.g., walking, running, biking, rowing). For example, the videographer 110a may use the video camera 106a to capture video while the videographer 110a runs in a real running race or along a running trail. A rendered image or video refers to an image or video generated by a game engine or rendering engine, such as the UNREAL ENGINE game engine, of a virtual world. For example, the wellness device system 100 may include a game engine 115 that may employed to render an image or video that may be used as a background image or video for combination with video of an instructor performing or directing a workout or a mental health improvement session. Additional details regarding combining video of an instructor with a background image or video are disclosed in U.S. Provisional Patent Application Ser. No. 63/156,801, filed Mar. 4, 2021, which is incorporated herein by reference in its entirety for all that it discloses.

In some embodiments, performance parameters of an instructor performing or directing a workout or mental health improvement session or of a videographer as the videographer captures video (e.g., to be used as background video) may be recorded as the instructor and/or videographer performs or directs the workout or mental health improvement session. For example, performance parameters may be recorded for the instructors 108a, 108b as they perform or direct their respective workouts or mental health improvement sessions and/or for the videographers 110a, 110b as they capture video while performing a workout. The performance parameters may include speed, cadence, heart rate, incline, or other performance parameters. Alternatively or additionally, a virtual speed of movement through a virtual environment depicted in a rendered video, an incline of the virtual environment, or other parameters of the rendered video or the virtual environment may be recorded. The performance parameters of the instructor and/or the videographer and/or the parameters of the rendered video or the virtual environment may be used to create wellness device control commands, as described in more detail elsewhere herein.

In some embodiments, video programs herein may include video or one or more images without an instructor in the video or images. For example, some video or images for use in video programs herein may depict real or virtual environments or scenery without an instructor, such as a beach, a mountain meadow, a field of flowers, a jungle, or other locations or objects devoid of an instructor. In some embodiments, such video may include audio of an instructor performing or directing a workout or a mental health improvement session without including the instructor in the video. For example, a video program for a mindfulness session may include video or images of one or more outdoor scenes with a voice (but no images or video) of an instructor directing the mindfulness session

The various videos discussed herein may be formatted in any one of multiple video formats, at least some of which being capable of supporting a subtitle stream. Some example formats may include MPEG-4, Dynamic Adaptive Streaming over HTTP (MPEG-DASH), and HTTP Live Streaming (HLS).

Next, a producer (not shown) or other user may utilize a computer 114 to input wellness device control commands for the video or the combined video into a video workout program or other video program, which may be encoded into a subtitle stream of the video or the combined video, or may be encoded separately from the video or the combined video, such as in separate data packets. For example, where the video or the combined video is being produced to be utilized as a live video workout program or other live video program, the producer may input the wellness device control commands using the computer 114 synchronously or substantially synchronously with the video camera 106b or 106c capturing the video of the instructor 108a, 108b performing or directing the workout (e.g., during a live event) and/or mental health improvement session and/or with generation of the combined video when one is generated. In this example, the producer may also give corresponding instructions to the instructor 108a, 108b, such as through an earpiece worn by the instructor 108a, 108b, to help the instructor 108a, 108b and the producer be in sync following a common script or plan for the workout or mental health improvement session. Alternatively, where the video or the combined video is produced to be utilized in a pre-recorded or archived video workout program or other archived video program, the producer may input wellness device control commands using the computer 114 subsequent to the capture of the video of the instructor 108a, 108b performing or directing the workout or mental health improvement session and/or generation of the combined video, where one is generated (e.g., minutes, hours, or days after the live event). The wellness device control commands may control operation of wellness devices at which the video workout program or other video program is executed.

In some embodiments, the producer may utilize the computer 114 to input output control commands into the video workout program or other video program, which may be encoded into the subtitle stream of the video or the combined video or may be encoded separately from the video or the combined video, such as in separate data packets. The output control commands may be input synchronously or substantially synchronously with the video camera 106b, 106c capturing the video of the instructor 108a, 108b performing or directing the workout or mental health improvement session and/or with generation of the combined video when one is generated. The output control commands may control operation of one or more output devices integrated with and/or in a vicinity of an exercise machine or other wellness device on which the video workout program or other video program is executed so as to control or affect an environment of a user of the exercise machine or other wellness device. Such output devices may include audio speakers, display devices, heat lamps, fans, oil diffusers, scent dispensers, lights, humidifiers, mist dispensers, or other output device. The output devices may be smart devices, may be communicatively coupled to a corresponding exercise machine or other wellness device, and/or may be communicatively coupled to the network 118, to receive the output control commands in the video workout program or other video program. An example output device is depicted in FIG. 1 as a sun lamp 119 in a vicinity of a wellness device 120a. Additional details regarding the generation of video workout programs with control commands that may be applied to the generation of video workout programs, video mental health programs, or other video programs as described herein can be found in U.S. patent application Ser. No. 16/742,762, filed Jan. 14, 2020 and U.S. Provisional Patent Application Ser. No. 63/156,801, filed Mar. 4, 2021, each of which is incorporated herein by reference in its entirety for all that it discloses.

In some embodiments, the video workout program or other video program, including the video or the combined video and the control commands (which may be encoded in the subtitle stream of the video or the combined video, or may be encoded separately from the video or the combined video) may then be transmitted over the network 118 from the remote server 112 in the remote location 102 to a local server 116 in the local location 104.

The video workout program or other video program may then be transmitted from the local server 116 to be used in connection with a wellness device 120a, 120b, 120c, or 120d. For example, the video workout program or other video program may be transmitted from the local server 116 to the wellness device 120a, 120b, 120c, or 120d, each of which may include a console 122, a touchscreen display, and/or other user interface. Alternatively or additionally, a separate tablet 124 may function as a console, or may function in connection with a console or other user interface, of the wellness device 120a, 120b, 120c, or 120d, and may also include a display, such as a touchscreen display. The tablet 124 may communicate with the console 122 and/or with the wellness device 120a, 120b, 120c, or 120d, via a network connection, such as a Bluetooth connection.

At the console 122 or the tablet 124, or more generally at the wellness device 120a, 120b, 120c, or 120d, the video or the combined video and the control commands (which may be encoded in the subtitle stream of the video or the combined video) may be decoded and/or accessed. Then, the console 122, the tablet 124, or more generally the wellness device 120a, 120b, 120c, or 120d may display the video or the combined video from the video workout program or other video program (e.g., of the instructor 108a, 108b performing or directing a workout or mental health improvement session) while simultaneously controlling one or more moveable members or output devices of the wellness device 120a, 120b, 120c, or 120d using the wellness device control commands and/or the output control commands. Additional details regarding controlling an exercise machine or environmental control device (which is an example of an output control device) using exercise machine control commands or environmental control commands (which are examples of output control commands) can be found in U.S. patent application Ser. No. 16/742,762, filed Jan. 14, 2020 and U.S. Provisional Patent Application Ser. No. 63/156,801, filed Mar. 4, 2021, each of which is incorporated herein by reference in its entirety for all that it discloses.

A user, such as a user 109, may perform a workout or mental health improvement session of a video program using the wellness device 120a, 120b, 120c, or 120d at which the video program is executed. Further, during performance of a workout or mental health improvement session by the user 109 using the video program on the wellness device 120a, 120b, 120c, or 120d, a heart rate of the user 109 may be monitored by the console 122, the tablet 124, or more generally the wellness device 122a, 122b, 122c, or 122d or other device. This heart rate monitoring may be accomplished by receiving continuous heart rate measurements wirelessly (such as over Bluetooth or Ant+) from a heart rate monitoring device worn by the user 109, such as a heart rate strap 111b or a heart rate watch 111a, or other wearable heart rate monitor. Alternatively, the heart rate monitoring device may be built into another device, such as being built into handlebars, handgrips, or other portion of the wellness device 120a, 120b, 120c, or 120d.

The heart rate strap 111b and the heart rate watch 111 are examples of sensors that may be used to generate and/or gather biological parameters, performance parameters, or other information of users of the wellness devices 120a, 120b, 120c, and/or 120d. Such sensors may generally include heart rate sensors (such as may be included in the heart rate strap 111b and the heart rate watch 111), VO2 max sensors, brain wave sensors, hydration level sensors, breathing/respiratory rate sensors, blood pressure sensors, current sensors, speed sensors (e.g., tachometers), weight sensors, pressure sensors, gait sensors, fingerprint sensors, biometric sensors (e.g., heart rate sensors, breathing sensors, gait sensors, fingerprint sensors), accelerometers, or other sensors. Such sensors may be integrated with, included in, coupled to, or otherwise associated with one or more of the wellness devices 120a, 120b, 120c, and/or 120d and/or the users of the wellness devices 120a, 120b, 120c, and/or 120d.

In some embodiments in which biological parameters are collected (such as heart rate), a probability that the biological data is accurate may be determined. For example, when gathering heart rate data from a heart-rate strap or heart rate watch (such as the heart rate strap 111b or the heart rate watch 111a) worn by the user, it is possible that the heart rate data is inaccurate due to improper positioning of the strap, some debris or other object or material blocking all or part of a sensor of the heart rate watch or strap, poor connectivity with the receiver, etc. To account for this possibility, some embodiments may analyze the probability of the heart rate data being accurate, and where the probability of accuracy is below some threshold may discard, ignore, or otherwise not rely on the heart rate data.

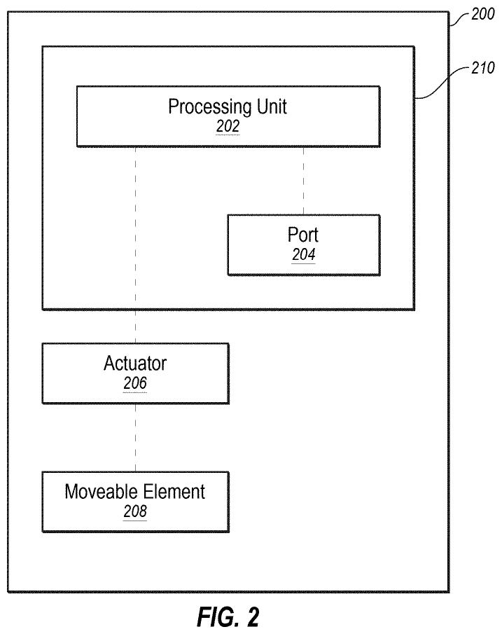

The wellness device 120a is illustrated in FIG. 1 as a treadmill. The treadmill 120a may include multiple different moveable members, including a running belt 126a and a running deck 126b, which may include one or more operating parameters that are selectively adjustable within a limited range. During performance of a workout or mental health improvement session using a video program on the treadmill 120a, the running belt 126a may rotate and the running deck 126b may incline. One example of an operating parameter on the treadmill 120a is a speed of the running belt 126a. The running belt 126a may rotate at different speeds within a limited range. An actuator (see FIG. 2), for example a belt motor, may selectively adjust the speed at which the running belt 126a rotates within the limited range. Another example of an operating parameter on the treadmill 120a is the inclination of running deck 126b. The running deck 126b may be selectively inclinable to different angles within a limited range. An actuator, for example an incline motor, may selectively adjust the incline of the running deck 126b within the limited range.

The wellness device 120b is illustrated in FIG. 1 as a sleep assistance device that may be placed, e.g., on a nightstand or other location on or near a bed of a user. The sleep assistance device 120b may include multiple different output devices, including a scent dispenser 128a, a display device in the form of a projector 128b, an audio speaker 128c, and a light 128d, which include one or more output parameters that may be controlled and/or adjusted as part of a sleep assistance session. During performance of the sleep assistance session using a video program on the sleep assistance device 120b, the projector 128b may be controlled to project one or more images and/or video (e.g., images and/or video of a soothing scene) onto a ceiling or wall or screen in a vicinity of the sleep assistance device 120b, the scent dispenser 128a may be controlled to output one or more scents (e.g., a soothing or restorative scent), the audio speaker 128c may be controlled to output one or more sounds, the light 128d may be controlled to output lighting (e.g., ambient lighting). The outputs of the scent dispenser 128a, the projector 128b, the audio speaker 128c, and the light 128d may be controlled by output control commands included in or accompanying the video program executed at the sleep assistance device 120b.

The wellness device 120c is illustrated in FIG. 1 as a smart yoga mat system. The smart yoga mat system 120c may include multiple different output devices, including multiple lights 128e distributed throughout a mat of the smart yoga mat system 120b, and an audio speaker, a display device, and a scent dispenser included in an electronics unit 128f coupled to the mat, the output devices each including one or more output parameters that may be controlled and/or adjusted as part of a yoga session. During performance of the yoga session using a video program on the smart yoga mat system 120b, the projector of the electronics unit 128f may be controlled to project one or more images and/or video (e.g., images and/or video of a soothing scene) onto a ceiling or wall or screen in a vicinity of the smart yoga mat system 120b, the scent dispenser of the electronics unit 128f may be controlled to output one or more scents (e.g., a soothing or restorative scent), the audio speaker of the electronics unit 128f may be controlled to output one or more sounds (e.g., instructions for one or more yoga poses), and the lights 128e may be controlled to illuminate portions of the yoga mat (e.g., to indicate where to place a hand or foot in a given yoga pose). The outputs of the lights 128e and the scent dispenser, the projector, and the audio speaker of the electronics unit 128f may be controlled by output control commands included in or accompanying the video program executed at the smart yoga mat system 120c.

The wellness device 120d is illustrated in FIG. 1 as an immersive mental health device. The immersive mental health device 120d may include one or more moveable members, including an adjustable chair 126c which may include one or more operating parameters that are selectively adjustable within a limited range. The immersive mental health device 120d may also include one or more output devices, such as a scent dispenser, a display device, an audio speaker, a heater, and a fan, which include one or more output parameters that may be controlled and/or adjusted as part of a mental health improvement session. During performance of the mental health improvement session using a video program on the immersive mental health device 120d, the adjustable chair 126c may be controlled to recline or otherwise position a user in a relaxed position, the scent dispenser may be controlled to output one or more scents (e.g., a soothing or restorative scent), the display device may be controlled to output one or more images and/or video (e.g., images and/or video of a soothing scene and/or a mindfulness instructor) on an interior of a housing of the immersive mental health device 120d, the audio speaker may be controlled to output one or more sounds (e.g., instructions for a mindfulness session), and the heater and/or fan may be controlled to maintain or adjust temperature in a vicinity of the user to a target temperature or target range of temperatures. The operating parameters of the adjustable chair 126c and the outputs of the scent dispenser, the display device, the audio speaker, the heater, and/or the fan may be controlled by wellness device control commands or output control commands included in or accompanying the video program executed at the immersive mental health device 120d.

FIG. 2 illustrates a block diagram of an example exercise machine or immersive mental health device 200. The exercise machine or immersive mental health device 200 of FIG. 2 is an example of a wellness device and is referred to hereinafter as wellness device 200. The wellness device 200 of FIG. 2 may represent, and may include similar components to, the treadmill 120a and/or the immersive mental health device 120d of FIG. 1. Alternatively or additionally, the wellness device 200 of FIG. 2 may represent, and may include similar components to, other exercise machines, such as an elliptical machine, an exercise bike, a rower machine, or other immersive mental health devices.

As disclosed in FIG. 2, the wellness device 200 may include a processing unit 202, a receiving port 204, an actuator 206, and a moveable member 208. The moveable member 208 may be similar to any of the moveable members 126a-126c of FIG. 1, for example. The processing unit 202 may be communicatively connected to the receiving port 204 and may be included within a console 210, which may be similar to the console 122 of FIG. 1, for example. The processing unit 202 may also be communicatively connected to the actuator 206. In response to control commands executed by the processing unit 202, the actuator 206 may selectively adjust one or more operating parameters of the moveable member 208 within a limited range.

Data, including data in a video workout program or a video mental health program, can be received by the wellness device 200 through the receiving port 204. As stated previously, a video workout program or video mental health program may include video as well as control commands. Control commands may provide control instructions to a wellness device (such as an exercise machine) and/or one or more associated output control devices. Control commands may include, for example, control commands for a belt motor, an incline motor, chair recline motor, and/or other actuators. In addition to actuator control commands, control commands may further include output control commands, distance control commands, time control commands, and/or heart rate zone control commands. These control commands may provide a series of actuator control commands or output control commands for execution at specific times or at specific distances. For example, a control command for an actuator to be at a certain level for a specific amount of time or for a specific distance or a control command for a sun lamp to output light at a certain level and/or with a certain spectral content for a specific amount of time. These control commands may also provide a series of actuator control commands or output control commands for execution at specific times or at specific distances based on a user's monitored heart rate, heart rate trends over time, other biometric parameters, mental state, responses to questions relating to mental health of the user, or the like. For example, a control command for an actuator may dictate a certain heart rate zone for a certain amount of time or distance, and a difficulty level of this control command may be dynamically scaled based on a user's monitored heart rate in order to get or keep the user in the certain heart rate zone for the certain amount of time or distance. Additional details regarding dynamically scaling a difficulty level of a control command based on a user's monitored heart rate can be found in U.S. patent application Ser. No. 16/742,762, filed Jan. 14, 2020, which is incorporated herein by reference in its entirety for all that it discloses. As another example, a control command for a sun lamp may dictate a brightness for a certain amount of time based on the user's mental state or responses to questions relating to the user's mental health.

Using a control command, received at the receiving port 204 in a video program, such as a control command that is decoded from a subtitle stream of a video of a video program for example, the processing unit 202 may control the actuator 206 or output device on or associated with the wellness device 200 in the sequence and at the times or distances specified by the control command. For example, actuator control commands that provide the processing unit 202 with commands for controlling a belt motor, an incline motor, a flywheel brake, stride length motor, a chair recline motor, or another actuator may be included in the control commands received in a video workout program at the wellness device 200.

Actuator control commands can be received for different time segments or distance segments of a workout or mental health improvement session. For example, a ten-minute workout or a ten-minute mindfulness session may have twenty different control commands that provide the processing unit 202 with a different control command for controlling an actuator or output device every thirty seconds. Alternatively, a ten-mile workout may have twenty different control commands that provide a processing unit with a different control command for controlling an actuator or output device every half mile. Workouts or mental health improvement sessions may be of any duration or distance and different control commands may be received at any time or distance during the workout or mental health improvement session. Alternatively, a 5-minute workout or mental health improvement session may have 300 different control commands that provide the processing unit 202 with a different control command for controlling an actuator or output device once per second.

The control commands received in a video program at the wellness device 200 may be executed by the processing unit 202 in a number of different ways. For example, the control commands may be received and then stored into a read/write memory that is included in or coupled to the processing unit 202. Alternatively, the control commands may be streamed to the wellness device 200 in real-time. The control commands may also be received and/or executed from a portable memory device, such as a USB memory stick or an SD card.

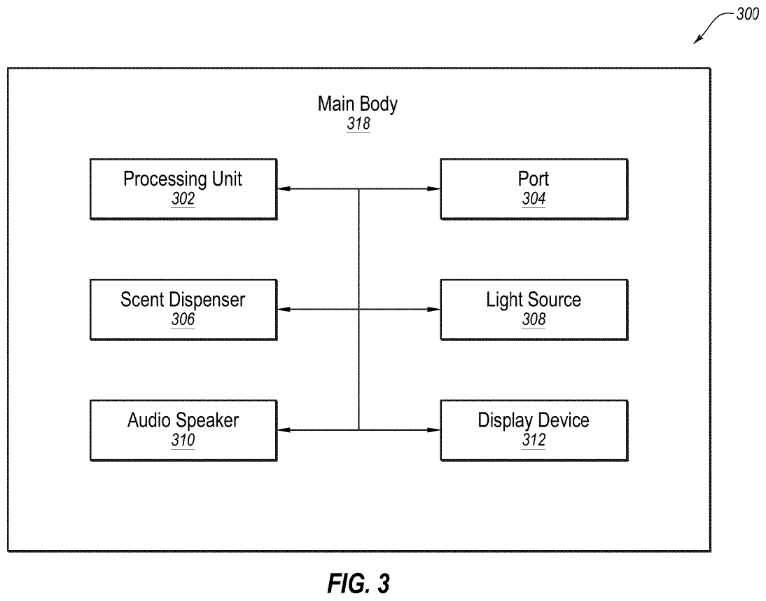

FIG. 3 illustrates a block diagram of an example sleep assistance device 300. The sleep assistance device 300 of FIG. 3 is an example of a wellness device. The sleep assistance device 300 of FIG. 3 may represent, and may include similar components to, the sleep assistance device 120b of FIG. 1. Alternatively or additionally, the sleep assistance device 300 of FIG. 3 may represent, and may include similar components to, other sleep assistance devices, some examples of which are described elsewhere herein.

As disclosed in FIG. 3, the sleep assistance device 300 may include a processing unit 302, a receiving port 304, and one or more output devices, including a scent dispenser 306, a light source 308, an audio speaker 310, and a display device 312 in this example. The output devices may be similar to corresponding output devices of FIG. 1, for example. The processing unit 302 may be communicatively connected, e.g., via a communication bus 314, to the receiving port 304 and may be supported in a main body 318 or housing of the sleep assistance device 300. The receiving port 304, the scent dispenser 306, the light source 308, the audio speaker 310, and the display device 312 may be supported in the main body 318. The processing unit 302 may also be communicatively connected to the scent dispenser 306, the light source 308, the audio speaker 310, and the display device 312. In response to control commands executed by the processing unit 302, the scent dispenser 306, the light source 308, the audio speaker 310, and/or the display device 312 may output one or more stimuli as part of a video program or other program. The sleep assistance device 300 may generally be configured to execute sleep assistance sessions configured to assist a user to reach and remain in a sleep state and/or to wake the user at a target time and/or after a target sleep duration.

Although not illustrated in FIG. 3, the sleep assistance device 300 may further include or be coupled to one or more sensors, such as a heart rate sensor, a respiratory rate sensor, or other sensor. Biological parameters of the user during sleep assistance sessions may be collected by the one or more sensors and may be used to generate new sleep assistance sessions for the user.

Data, including data in a video mental health program that includes or embodies a sleep assistance session, can be received by the sleep assistance device 300 through the receiving port 304. As stated previously, a video mental health program may include video as well as control commands. Control commands may provide control instructions to a wellness device (such as a sleep assistance device) and/or one or more associated output devices. Control commands may include, for example, control commands for a light source, a scent dispenser, a display device, an audio speaker, or other output device. These control commands may provide a series of output control commands for execution at specific times. For example, a control command for a scent dispenser to output scent at a certain level for a specific amount of time. These control commands may also provide a series of output control commands for execution at specific times based on a user's monitored heart rate, heart rate trends over time, other biometric parameters, mental state, responses to questions relating to mental health of the user, or the like. For example, a control command for a sun lamp may dictate a brightness for a certain amount of time based on the user's mental state or responses to questions relating to the user's mental health.

Using a control command, received at the receiving port 304 in a video program, such as a control command that is decoded from a subtitle stream of a video of a video program for example, the processing unit 302 may control the scent dispenser 306, the light source 308, the audio speaker 310, the display device 312, and/or other output device (such as a haptic device) in the sequence and at the times specified by the control command.

Output control commands can be received for different time segments or distance segments of a sleep assistance session. For example, a ten-minute sleep assistance session may have twenty different control commands that provide the processing unit 302 with a different control command for controlling an output device every thirty seconds. Sleep assistance sessions may be of any duration and different control commands may be received at any time or distance during the sleep assistance session. Alternatively, a 5-minute sleep assistance session may have 300 different control commands that provide the processing unit 302 with a different control command for controlling an output device once per second.

The control commands received in a video program at the sleep assistance device 300 may be executed by the processing unit 302 in a number of different ways. For example, the control commands may be received and then stored into a read/write memory that is included in or coupled to the processing unit 302. Alternatively, the control commands may be streamed to the sleep assistance device 300 in real-time. The control commands may also be received and/or executed from a portable memory device, such as a USB memory stick or an SD card.

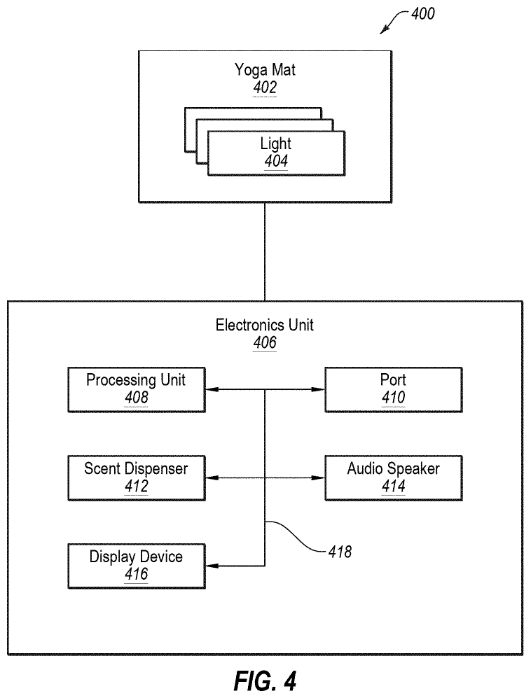

FIG. 4 illustrates a block diagram of an example smart yoga mat system 400. The smart yoga mat system 400 of FIG. 4 is an example of a wellness device. The smart yoga mat system 400 of FIG. 4 may represent, and may include similar components to, the smart yoga mat system 120c of FIG. 1. Alternatively or additionally, the smart yoga mat system 400 of FIG. 4 may represent, and may include similar components to, other smart yoga mat systems.

As disclosed in FIG. 4, the smart yoga mat system 400 may include a yoga mat 402 with lights 404 distributed throughout the yoga mat and an electronics unit 406 coupled to the yoga mat 402. The electronics unit 406 may include a processing unit 408, a receiving port 410, and one or more output devices, including a scent dispenser 412, an audio speaker 414, and a display device 416 in this example. The output devices may be similar to corresponding output devices of FIG. 1, for example. Although not illustrated in FIG. 4, the smart yoga mat system 400 may further include a sensor, such as a video camera spaced apart from the yoga mat or pressure sensors integrated in the yoga mat 402, to monitor poses of a user during performance of yoga sessions. The processing unit 408 may be communicatively connected, e.g., via a communication bus 418, to the receiving port 410 and may be supported in the electronics unit 406. The receiving port 410, the scent dispenser 412, the audio speaker 414, and the display device 416 may be supported in the electronics unit 406. The processing unit 408 may also be communicatively connected to the scent dispenser 412, the audio speaker 414, and the display device 416. In response to control commands executed by the processing unit 408, the lights 404, the scent dispenser 412, the audio speaker 414, and/or the display device 416 may output one or more stimuli as part of a video program or other program. The smart yoga mat system 400 may generally be configured to execute yoga sessions for users.

Data, including data in a video mental health program that includes or embodies a yoga session, can be received by the smart yoga mat system 400 through the receiving port 410. As stated previously, a video mental health program may include video as well as control commands. Control commands may provide control instructions to a wellness device (such as a smart yoga mat system) and/or one or more associated output devices. Control commands may include, for example, control commands for a scent dispenser, a display device, an audio speaker, or other output device. These control commands may provide a series of output control commands for execution at specific times. For example, a control command for a scent dispenser to output scent at a certain level for a specific amount of time. These control commands may also provide a series of output control commands for execution at specific times based on a user's monitored heart rate, heart rate trends over time, other biometric parameters, mental state, responses to questions relating to mental health of the user, sensor feedback regarding the user's poses, or the like. For example, a control command for the lights 404 may light up a subset of the lights 404 to show the user where to place one or both of the user's hands and/or feet for a given pose.

Using a control command, received at the receiving port 410 in a video program, such as a control command that is decoded from a subtitle stream of a video of a video program for example, the processing unit 408 may control the lights 404, the scent dispenser 412, the audio speaker 414, the display device 416, and/or other output device (such as a haptic device) in the sequence and at the times specified by the control command.

Output control commands can be received for different time segments of a yoga session. For example, a ten-minute yoga session may have twenty different control commands that provide the processing unit 408 with a different control command for controlling an output device every thirty seconds. Yoga sessions may be of any duration and different control commands may be received at any time during the yoga session. Alternatively, a 5-minute yoga session may have 300 different control commands that provide the processing unit 408 with a different control command for controlling an output device once per second.

The control commands received in a video program at the smart yoga mat system 400 may be executed by the processing unit 408 in a number of different ways. For example, the control commands may be received and then stored into a read/write memory that is included in or coupled to the processing unit 408. Alternatively, the control commands may be streamed to the smart yoga mat system 400 in real-time. The control commands may also be received and/or executed from a portable memory device, such as a USB memory stick or an SD card.

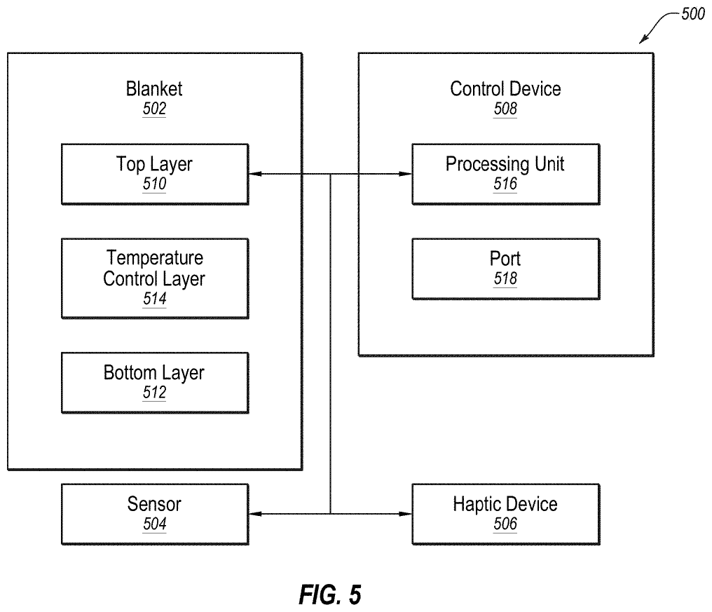

FIG. 5 illustrates a block diagram of an example smart blanket 500. The smart blanket 500 of FIG. 5 is an example of a wellness device and specifically of a sleep assistance device. The smart blanket 500 of FIG. 5 may include at least some similar components to the sleep assistance device 120b of FIG. 1. Alternatively or additionally, the smart blanket 500 of FIG. 5 may represent, and may include similar components to, other smart blankets.

As disclosed in FIG. 5, the smart blanket 500 may include a blanket 502, a sensor 504, a haptic device 506, and/or a control device 508. The blanket 502 may be a weighted blanket and/or may include one or more layers, such as a top layer 510, a bottom layer 512, and a temperature control layer 514 positioned between the top and bottom layers 510, 512. The control device 508 may include a processing unit 516 and a receiving port 518. The processing unit 516 may be communicatively connected to the temperature control layer 514, the sensor 504, the haptic device 506, and/or the port 518, e.g., via a communication bus 520. In response to control commands executed by the processing unit 516, the temperature control layer 514 and/or the haptic device 506 may output one or more stimuli as part of a video program or other program. The smart blanket 500 may generally be configured to execute sleep assistance sessions for users.

Data, including data in a video or audio mental health program that includes or embodies a sleep assistance session, can be received by the smart blanket 500 through the receiving port 518. A video or audio mental health program may include video and/or audio as well as control commands. Control commands may provide control instructions to a wellness device (such as a smart blanket) and/or one or more associated output devices. Control commands may include, for example, control commands for a temperature control layer, a haptic device, or other output device. These control commands may provide a series of output control commands for execution at specific times. For example, a control command for a temperature control layer to control temperature to a target temperature or target range of temperatures for a specific amount of time. These control commands may also provide a series of output control commands for execution at specific times based on a user's monitored heart rate, heart rate trends over time, other biometric parameters, mental state, responses to questions relating to mental health of the user, or the like. For example, a control command for the haptic device 506 may cause the haptic device 506 to vibrate at a certain frequency or with a certain duty cycle to bring a respiratory rate of a user as sensed by the sensor 504 to the frequency.

Using a control command, received at the receiving port 518 in or with a video or audio program, such as a control command that is decoded from a subtitle stream of a video of a video program for example, the processing unit 516 may control the temperature control layer 514, the haptic device 506, and/or other output device in the sequence and at the times specified by the control command.

Output control commands can be received for different time segments of a sleep assistance session. For example, a ten-minute sleep assistance session may have twenty different control commands that provide the processing unit 408 with a different control command for controlling an output device every thirty seconds. Sleep assistance sessions may be of any duration and different control commands may be received at any time during the sleep assistance session. Alternatively, a 5-minute sleep assistance session may have 300 different control commands that provide the processing unit 408 with a different control command for controlling an output device once per second.

The control commands received in or with a video or audio program at the smart blanket 500 may be executed by the processing unit 408 in a number of different ways. For example, the control commands may be received and then stored into a read/write memory that is included in or coupled to the processing unit 408. Alternatively, the control commands may be streamed to the smart blanket 500 in real-time. The control commands may also be received and/or executed from a portable memory device, such as a USB memory stick or an SD card.

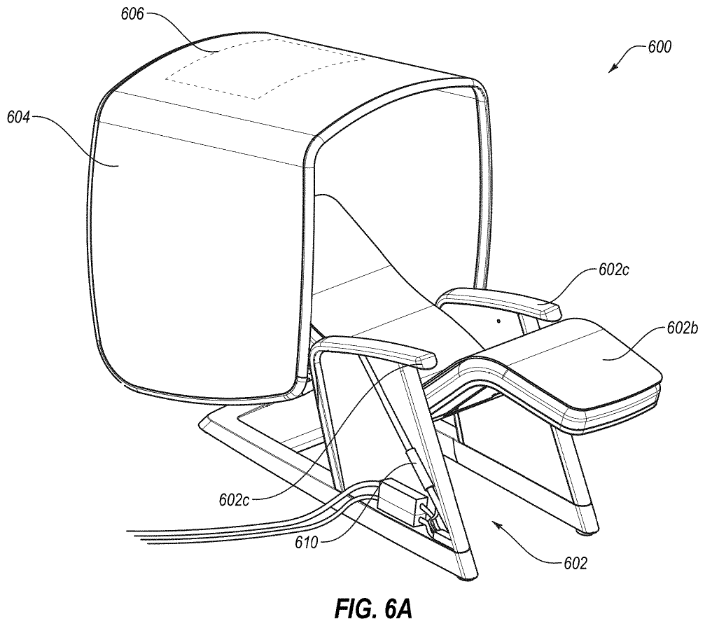

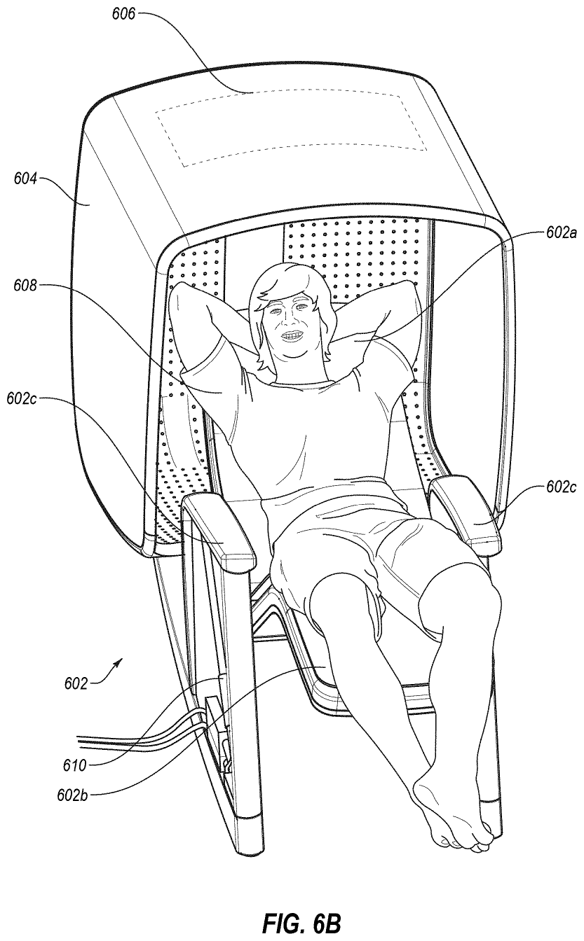

FIGS. 6A and 6B illustrate perspective views of an example immersive mental health device 600. The immersive mental health device 600 of FIGS. 6A and 6B is an example of a wellness device. The immersive mental health device 600 of FIGS. 6A and 6B may represent, and may include similar components to, the immersive mental health device 120d of FIG. 1 and/or the wellness device 200 of FIG. 2. Alternatively or additionally, the immersive mental health device 600 of FIGS. 6A and 6B may represent, and may include similar components to, other immersive mental health devices.

As disclosed in FIGS. 6A and 6B, the immersive mental health device 600 may include an adjustable chair 602, a housing 604, and a display device 606, among potentially other components (e.g., such as those discussed with respect to FIG. 2). FIG. 6A shows the housing 604 in a partially closed position and FIG. 6B shows the housing 604 in an open position with a user 608 seated in the adjustable chair 602. In FIGS. 6A and 6B, the display device 606 is shown as a dashed outline at an approximate location of where the display device 606 may be located inside the housing 604. In practice, the display device 606 may not be visible from outside the housing 604 as the housing 604 may be opaque to at least partially isolate the user 608 from external distractions (e.g., any distractions external to the housing 604).

The display device 606 may include a flat-panel monitor or television or other emissive display as illustrated in FIGS. 6A and 6B, a projector to project images and/or video onto an interior surface of the housing 604 in front of the face of the user 608, or other suitable display device. In some embodiments, a display area coverage (whether emissive or projected) of the display device 606 on the interior surface of the housing 604 may be panoramic, 120 degrees, 160 degrees, or other amount. When a projector is implemented as the display device 606, it may be mounted to a head rest 602a of the adjustable chair 602, an interior surface of the housing 604, or other structure of the immersive mental health device 600. The display device 606 implemented as the flat-panel monitor or television may have an audio speaker integrated therewith. Alternatively or additionally, an audio speaker may be included in the immersive mental health device 600 separate from the display device 606. The display device 606 may be coupled to an inner surface of the housing 604 and positioned to be in view of the user when the housing 604 is in the closed position or may be positioned to project image or video content onto the inner surface of the housing 604 at a location in view of the user when the housing 604 is in the closed position.

The adjustable chair 602 may include one or more movable components. For example, one or more of the head rest 602a, a footrest 602b, arm rests 602c, or other components of the adjustable chair 602 may be movable. The head rest 602a, the footrest 602b, the arm rests 602c, and/or other components may be movable independent of each other or together. The adjustable chair 602 may include one or more actuators 610 to effect movement of the head rest 602a, the footrest 602b, the arm rests 602c, and/or other components of the adjustable chair 602.

Alternatively or additionally, the adjustable chair 602 and/or the housing 604 may include or have coupled thereto one or more compression members, haptic devices, heater elements, cooler elements, humidity control elements or other components to output tactile stimuli to the user 608 and/or to control an environment of the user 608 within the housing 604. Each compression member may include a partial or whole sleeve or channel to accommodate all or a portion of a trunk, limb (e.g., arm, leg), extremity (e.g., hand, finger, foot, toe), or other body part of the user 608 and which may compress around or from opposing sides of the body part as, e.g., massage, to promote blood flow, or for other purpose. Each haptic device may be configured to vibrate or provide other tactile output as, e.g., massage, to adjust respiratory rate and/or heart rate of the user 608, or for other purpose. Each heater and cooler element may be configured to respectively heat or cool the user 608, a portion of the user 608, and/or the environment within the housing 604. Each humidity control element may include a humidifier, a dehumidifier, or other device or system to control humidity of the environment within the housing 604.

While not illustrated in FIGS. 6A and 6B, the immersive mental health device 600 may include or be coupled to one or more sensors to collect biological parameters of the user 608. In some embodiments, the one or more sensors may be configured to detect a maximal oxygen uptake, or VO2 max, of the user 608. Because the housing 602 substantially encloses the user 608 when in the closed position, an amount of oxygen coming in and out of the enclosed space, which is driven by the user 608 breathing, may be detected without placing a VO2 max sensor directly on the user 608. Alternatively or additionally, the adjustable chair 602 may include a scale to weigh the user 608 each time the user 608 is in the adjustable chair 602.

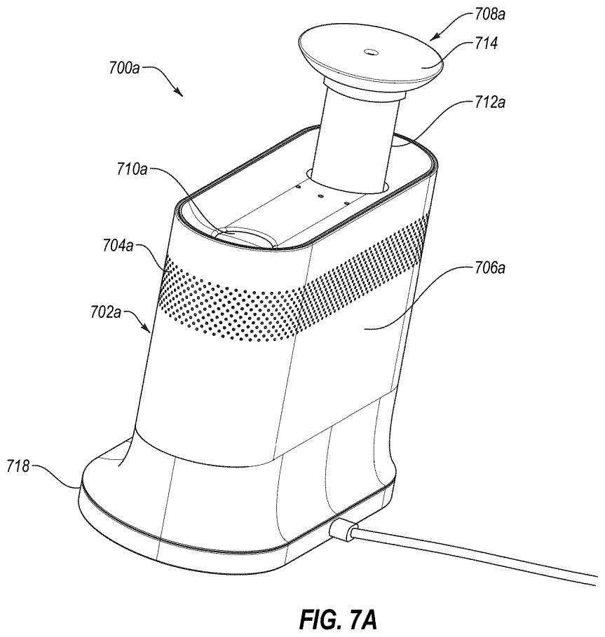

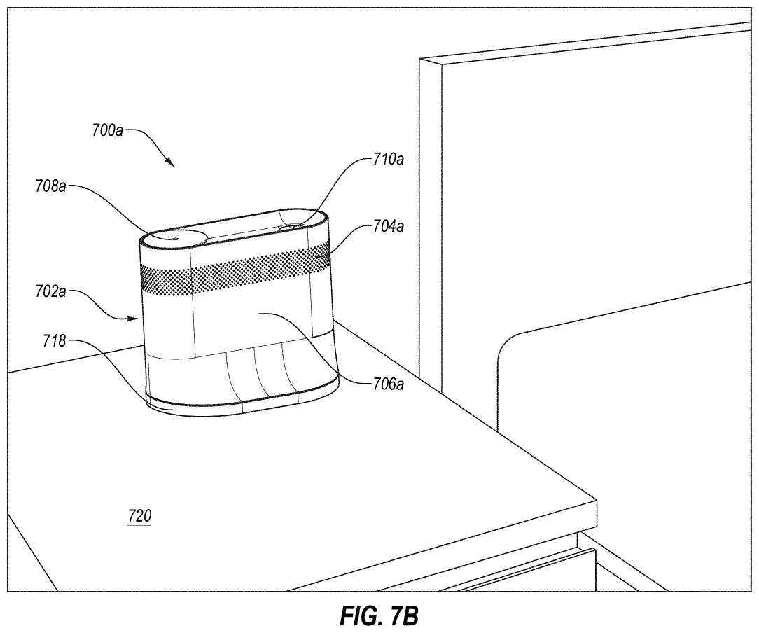

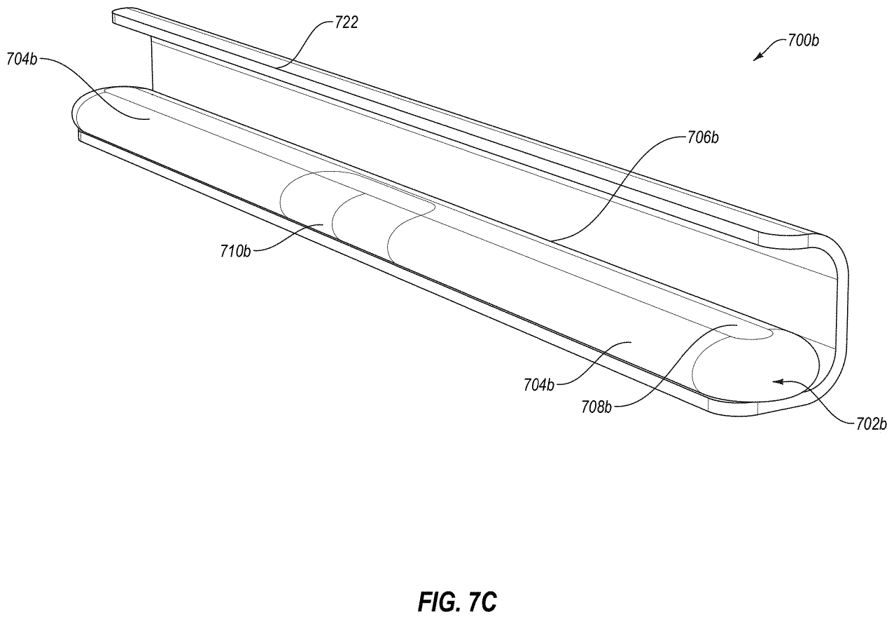

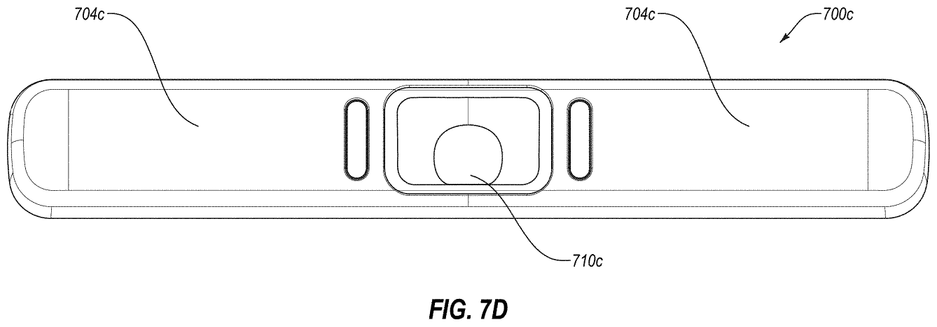

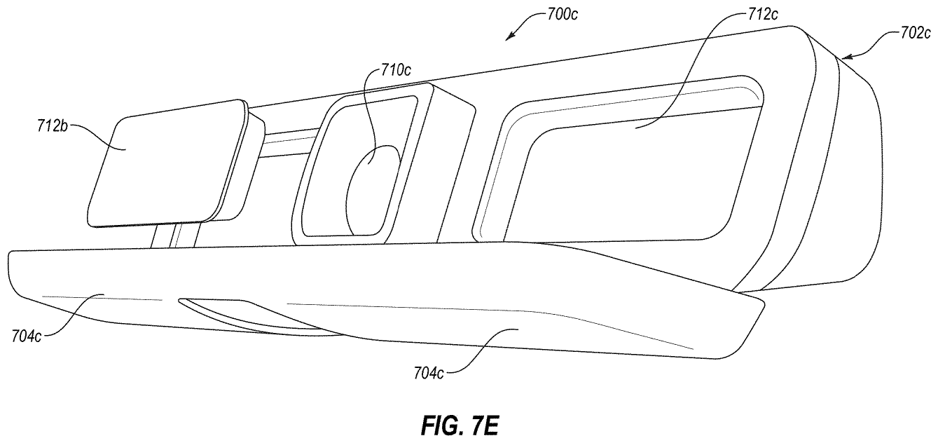

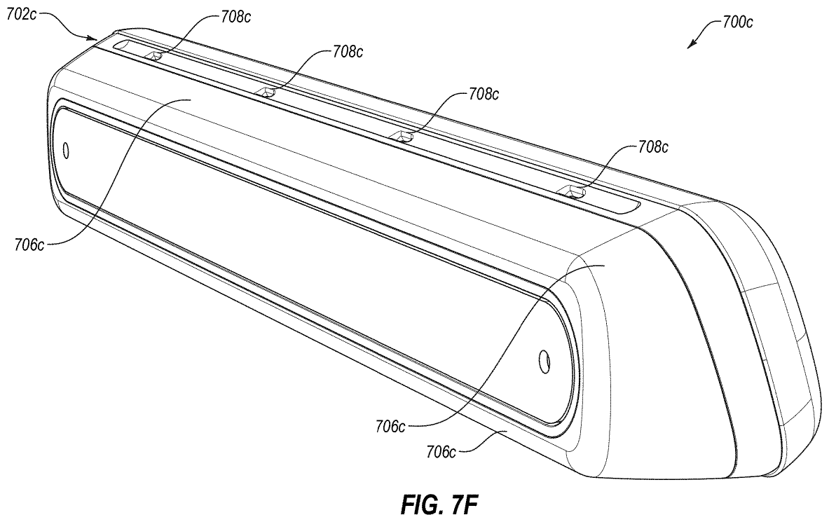

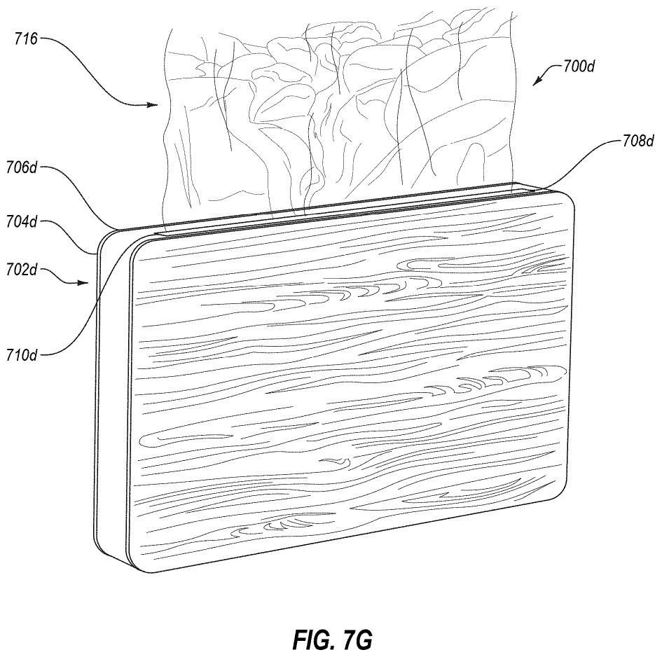

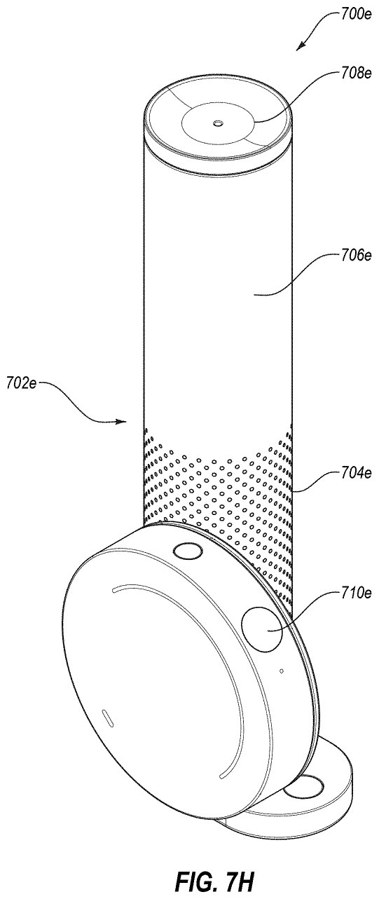

FIGS. 7A-7H illustrate perspective views of various example sleep assistance devices 700a, 700b, 700c, 700d, and 700e (hereinafter collectively “sleep assistance devices 700a-700e”). The sleep assistance devices 700a-700e of FIGS. 7A-7H are examples of wellness devices. The sleep assistance devices 700a-700e of FIGS. 7A-7H may represent, and may include similar components to, the sleep assistance device 120b of FIG. 1 and/or the sleep assistance device 300 of FIG. 3. Alternatively or additionally, the sleep assistance devices 700a-700e of FIGS. 7A-7H may represent, and may include similar components to, other sleep assistance devices.

As disclosed in FIGS. 7A-7H, each of the sleep assistance devices 700a-700e may include a main body or housing 702a, 702b, 702c, 702d, and 702e (hereinafter collectively “main bodies 702a-702e”), a processing unit (not shown) supported in the main bodies 702a-702e, an audio speaker 704a, 704b, 704c, 704d, and 704e (hereinafter collectively “audio speakers 704a-704e”), a light source 706a, 706b, 706c, 706d, and 706e (hereinafter collectively “light sources 706a-706e”), a scent dispenser 708a, 708b, 708c, 708d, and 708e (hereinafter collectively “scent dispensers 702a-702e”), and a display device 710a, 710b, 710c, 710d, and 710e (hereinafter collectively “display devices 710a-710e”). Each of the audio speakers 704a-704e, light sources 706a-706e, scent dispensers 708a-708e, and/or display devices 710a-710e may be supported in the corresponding main body 702a-702e and may be communicatively coupled to the corresponding processing unit.

One or more of the audio speakers 704a-704e, light sources 706a-706e, scent dispensers 708a-708e, and display devices 710a-710e may be supported or retained within an interior of a corresponding one of the main bodies 702a-702e. Lead lines for such output devices in FIGS. 7A-7H may indicate approximately where on the main bodies 702a-702e an output of the corresponding output device may be output from the main bodies 702a-702e and/or perceived by a user. In some embodiments, the main bodies 702a-702e may be transparent, porous, and/or formed with one or more openings to accommodate output from any output devices therein.

Each of the audio speakers 704a-704e may be configured to output audio stimuli configured to help a user reach and remain in a sleep state (e.g., at night) and/or to wake up from the sleep state (e.g., in the morning), such as soothing music, nature sounds, instructions or other commentary from an instructor, or other audio stimuli.

Each of the light sources 706a-706e may be configured to output visual stimuli that may aid a user to reach and remain in a sleep state (e.g., at night) and/or to wake up from the sleep state (e.g., in the morning). In some embodiments, each of the light sources 706a-706e may output a soft ambient light. Each of the light sources 706a-706e may emit light of a particular wavelength or range of wavelengths and/or may have an adjustable range of operating wavelengths and/or intensities. Alternatively or additionally, each of the light sources 706a-706e may include multiple light sources, each configured to emit light in a fixed or adjustable range of wavelengths. Some wavelengths of light, such as red wavelengths (e.g., about 620 nanometers (nm) to 750 nm), may promote healing, e.g., at a cellular level, and/or may stimulate production of melatonin to aid in reaching a sleep state. Some wavelengths of light, such as blue wavelengths (e.g., about 450 nm to 495 nm), may suppress onset of melatonin and/or increase alertness. Other wavelengths of light induce other effects in humans. For example, infrared (IR) light (e.g., about 750 nm to 1 millimeter (mm)) is generally not visible to humans but can be felt by humans as heat. Accordingly, each of the light sources 706a-706e may be fixed at or adjustable to one or more target wavelengths (or wavelength ranges) of light that may induce a desired effect in humans, which effects in some embodiments may aid a user in reaching and remaining in a sleep state and/or awaking from the sleep state. For example, when helping a user reach a sleep state, the light sources 706a-706e may output light that simulates light from a sunset and that may change from lighter to darker with corresponding change in color from more white light (or less red light) to more red light and eventually no or little light. When helping a user awake from a sleep state, the light sources 706a-706e may output light that simulates a sunrise and that may change from darker to lighter with corresponding change in color.

Each of the scent dispensers 708a-708e may be configured to output olfactory stimuli configured to help a user reach and remain in a sleep state (e.g., at night) and/or to wake up from the sleep state (e.g., in the morning). In some embodiments, each of the scent dispensers 708a, 708e may include a diffuser coupled to one or more scent cartridges such as disclosed in FIGS. 7A and 7E. In more detail, FIG. 7A is a partially exploded view of the sleep assistance device 700a which shows a scent cartridge 712a coupled to a diffuser 714, the scent cartridge 712a and the diffuser 714 being positioned at least partially within the main body 702a in operation. FIG. 7E is a partially exploded view of the sleep assistance device 700c which shows scent cartridges 712b, 712c each of which may be coupled to the same diffuser or different diffusers (not shown in FIG. 7E) of the sleep assistance device 700c, where the scent cartridges 712b, 712c may be positioned at least partially within the main body 702c in operation.

The scent cartridges such as 712a-712c that may be implemented according to embodiments herein may be disposable, refillable, recycleable, and/or biodegradable. In some embodiments, a given scent cartridge may have multiple discrete compartments, each of which has a different scent so that multiple scents may be dispensed from a single scent cartridge individually and/or together. In some embodiments, a supplier of scent cartridges may release one or more new scents on a monthly or other basis and users may optionally subscribe to receive one or more new scents on a monthly or other basis.

The scent or scents included in each scent cartridge may include any desired scent. Some scents, such as a lavender, may aid a user to reach and remain in a sleep state and/or to wake up from the sleep state. Accordingly, in some embodiments, one or more of the scent dispensers 708a-708e may include lavender or other scents.

Each of the display devices 710a-710e may be configured to output visual stimuli configured to help a user reach and remain in a sleep state (e.g., at night) and/or to wake up from the sleep state (e.g., in the morning). In some embodiments, each of the display devices 710a-710e may include a projector (e.g., a standard projector or an Ultra Short Throw (UST) projector), a flat-panel monitor or television, a vapor display, and/or other suitable display device. Each of the display devices 710a-710e of FIGS. 7A-7H is depicted as a projector that may output images and/or video on a nearby wall, ceiling, or other surface or objects. In the example of FIG. 7G, the display device 710d is specifically a vapor display in which water vapor 716 or other vapor is emitted from the sleep assistance device 700d and the display device 710d, e.g., the projector, projects images or video onto the water vapor 716.

The display devices 710a-710e may output images, video, and/or light (such as the light sources 706a-706e) to help the user reach and remain in a sleep state and/or to wake up from the sleep state. For example, the display devices 710a-710e may output calming or soothing images or video (e.g., of nature scenes, night sky, sunsets, sunrises), images or video of an instructor directing the user in relaxation or other techniques, or other images or video.

The sleep assistance device 700a of FIG. 7A may additionally include a charging dock 718. The sleep assistance device 700a may include one or more batteries that may charge when the sleep assistance device 700a is connected to the charging dock 718 and may be used to power the sleep assistance device 700a when disconnected from the charging dock 718. As such, the sleep assistance device 700a may easily be carried from room to room, on vacation, or may otherwise be easily portable. One or more of the other sleep assistance devices 700b-700e may similarly include a charging docket and one or more batteries to permit portable and/or unplugged operation. FIG. 7B shows the sleep assistance device 700a placed on a nightstand 720 next to the user's bed so that the sleep assistance device 700b may be used as an aid for the user to reach and maintain a sleep state or awake from a sleep state.

The sleep assistance device 700b of FIG. 7C may additionally include shelf 722. The shelf 722 may be mounted to a wall or other structure, e.g., on or above a headboard of the user's bed so that the sleep assistance device 700b may be used as an aid for the user to reach and maintain a sleep state or awake from a sleep state. In some embodiments, the shelf 722 may also serve as a light reflector to reflect light emitted from the light source 706b, which is shown as being located on a rear of the main body 702b in the example of FIG. 7C.

The sleep assistance device 700c of FIGS. 7D-7F may, similar to the sleep assistance device 700b of FIG. 7C, be mounted to a wall or other structure, e.g., on or above a headboard of the user's bed so that the sleep assistance device 700c may be used as an aid for the user to reach and maintain a sleep state or awake from a sleep state.

The sleep assistance device 700d of FIG. 7G may be positioned at or coupled to a foot or footboard of the user's bed so that the sleep assistance device 700d may be used as an aid for the user to reach and maintain a sleep state or awake from a sleep state. While illustrated and described as having a vapor display 710d, other implementations of sleep assistance devices may have retractable flat-panel television or monitor displays that may be retracted within the main body 702d when not in use and may be deployed out of the main body 702d during use.

One or more of the sleep assistance devices 700a-700e may include a user interface with one or more buttons, touchscreens, touch surfaces, microphones, or other input elements. Alternatively or additionally, users may control the sleep assistance devices 700a-700e wireless using a corresponding app or application running on a smartphone, tablet, or other electronic device.

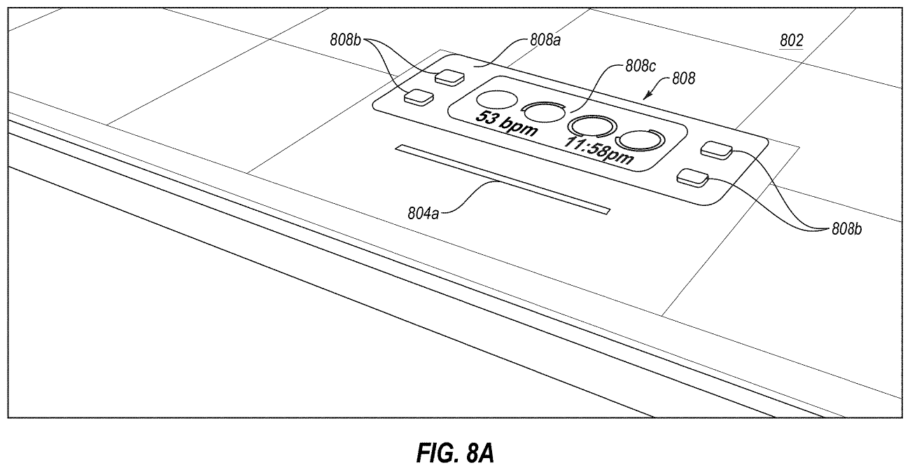

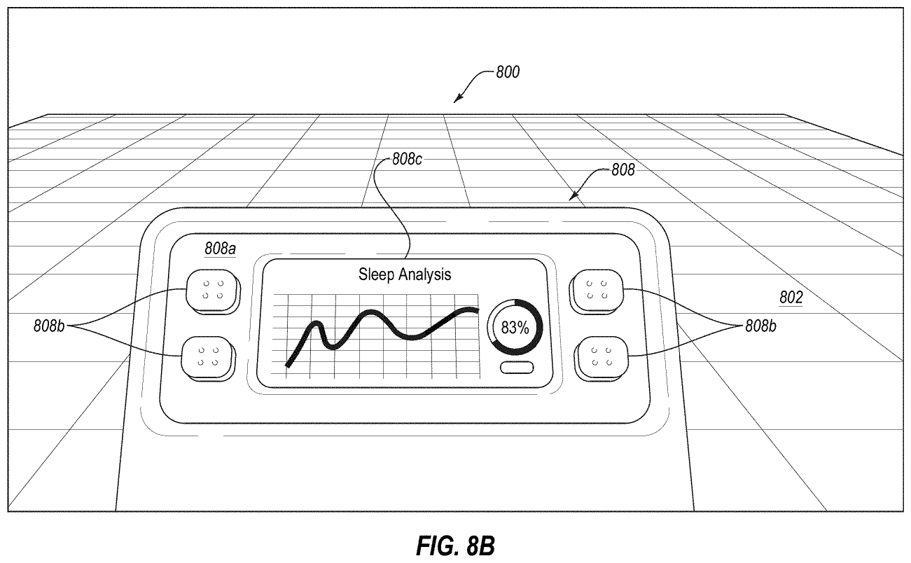

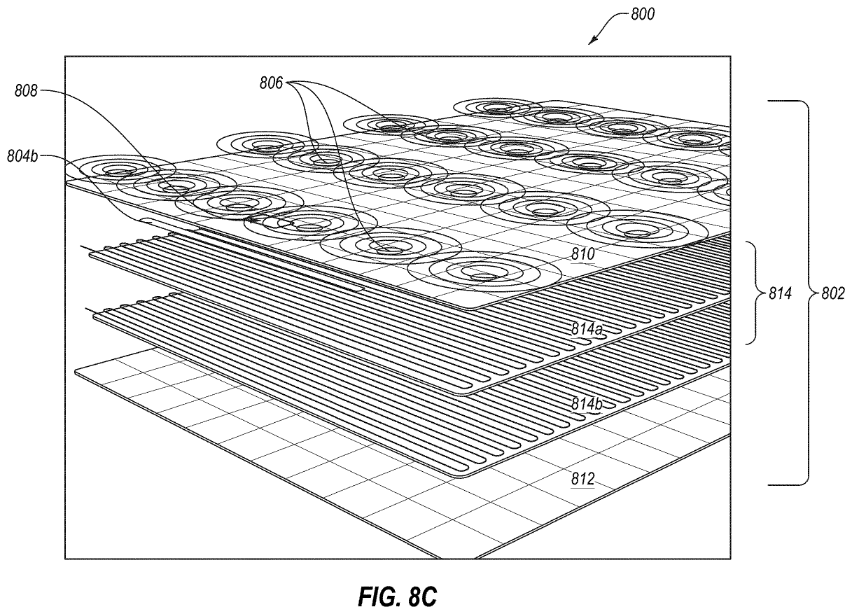

FIGS. 8A-8C illustrate various views of an example smart blanket 800. The smart blanket 800 of FIGS. 8A-8C is an example of a wellness device. The smart blanket 800 of FIGS. 8A-8C may represent, and may include similar components to, the smart blanket 500 of FIG. 5. Alternatively or additionally, the smart blanket 800 of FIGS. 8A-8C may represent, and may include similar components to, other smart blankets.

As disclosed in FIGS. 8A-8C, the smart blanket 800 may include a blanket 802, one or more sensors 804a (FIG. 8A), 804b (FIG. 8C) coupled to the blanket 802, one or more haptic devices 806 (FIG. 8C) (only some are labeled for simplicity) coupled to the blanket 802, and a control device 808 coupled to the blanket 802.

Referring to FIGS. 8A and 8B, the control device 808 may include a user interface 808a with one or more output and/or input devices. As illustrated, for example, the user interface 808a may include input buttons 808b to accept input and a display 808c to provide output to the user. Alternatively or additionally, the display 808c may include a touchscreen display to accept input. In some embodiments, the control device 808 may collect respiratory rate, heart rate, and/or other parameters of the user during use of the smart blanket, e.g., while the user is sleeping, and may present a graph to the user such as that illustrated on the display 808c in FIG. 8B that shows how one or more parameters varied while the user slept.

Referring to FIG. 8C, the blanket 502 may be a weighted blanket and/or may include one or more layers, such as a top layer 810, a bottom layer 812, and a temperature control layer 814 positioned between the top and bottom layers 810, 812.

The temperature control layer 814 may include a heater sublayer 814a and a cooler sublayer 814b. The heater sublayer 814a may include, for example, electrical heating wires. Passing current through the electrical heating wires may generate heat due to resistance of the electrical heating wires, which heat may warm the user. The cooler sublayer 814b may include, for example, one or more coolant conduits coupled to a coolant source. Circulating coolant through the conduits may absorb heat from the user to cool the user. In some embodiments, the temperature control layer 814 may include a conduit with vents through the bottom layer 812 and the smart blanket 800 may further include a control box with a fan and a heater element and a hose coupled between the control box and the temperature control layer. The heater element may be configured to generate heated air. The fan may be configured to circulate the heated air or air at room temperature through the temperature control layer 814 and out the vents of the bottom layer 812. Circulating heated air out the vents may warm a user under the smart blanket 800. Circulating air at room temperature out the vents may cool a user under the smart blanket 800 through evaporation cooling.

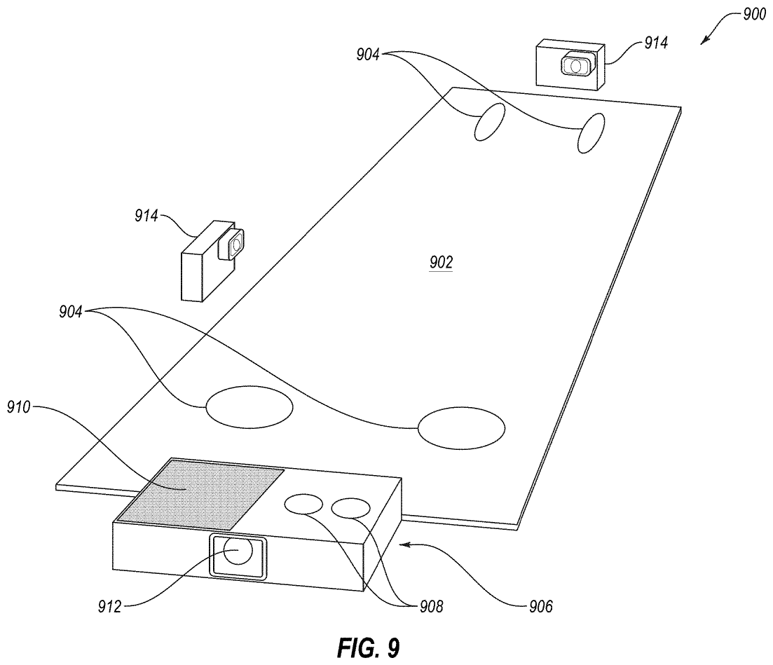

FIG. 9 illustrates an example smart yoga mat system 900. The smart yoga mat system 900 of FIG. 9 is an example of a wellness device. The smart yoga mat system 900 of FIG. 9 may represent, and may include similar components to, the smart yoga mat system 120c of FIG. 1 and/or the smart yoga mat system 400 of FIG. 4. Alternatively or additionally, the smart yoga mat system 900 of FIG. 9 may represent, and may include similar components to, other smart yoga mat systems.

As disclosed in FIG. 9, the smart yoga mat system 900 may include a yoga mat 902 with lights 904 distributed throughout the yoga mat 902 and an electronics unit 906 coupled to the yoga mat 902. In FIG. 9, only a subset of the lights 904 are turned on and visible; the yoga mat 902 may include additional lights 904 distributed throughout that are not visible in FIG. 9 because they are turned off.

The electronics unit 906 may include, e.g., a processing unit (not visible) and one or more output devices, including scent dispensers 908, an audio speaker 910, and a display device 912 in this example. The scent dispensers 908, the audio speaker 910, and the display device 912 may be supported in or by the electronics unit 906 or a main body or housing thereof and may be communicatively coupled to the processing unit of the electronics unit 906. The output devices may be similar to corresponding output devices of FIGS. 1 and 4, for example.

As illustrated in FIG. 9, the smart yoga mat system 900 may further include one or more sensors, such as video cameras 914, spaced apart from the yoga mat 902 or integrated within the electronics unit 906. Alternatively or additionally, the smart yoga mat system 900 may further include one or more pressure sensors integrated in the yoga mat 402 The sensors may be configured to monitor poses of a user during performance of yoga sessions with the smart yoga mat system 900. For example, the video cameras 914 may capture one or more images or video of the user in one or more yoga poses and/or the pressure sensors may determine positioning of one or more contact points (e.g., hands, feet, shoulders, head, elbows, etc.) of the user with the yoga mat 902. The images or video and/or the pressure sensor data may be processed (e.g., locally at the electronics unit 906 or at a remote device) to determine whether the user is performing a yoga pose correctly. For example, the images or video and/or the pressure sensor data may be processed to extract a model of the user in the yoga pose and the model of the user may be compared to a target model for the pose. If the user is doing the yoga pose incorrectly, the smart yoga mat system 900 may provide feedback to the user to correct the yoga pose. For example, the lights 904 may be selectively turned on to identify positions for one or more contact points of the user with the yoga mat. In the example of FIG. 9, the lights 904 show positioning of hands and feet for the downward facing dog yoga pose. As another example, the display device 912 may output an image or video of the target pose, an instructor in the target pose, or the like. Optionally, such an image or video may be combined with an overlay of the model of the user so the user can easily see aspects of the yoga pose of the user that are incorrect. As still another example, the audio speaker 910 may output instructions from an instructor regarding generally how to properly perform the yoga pose or specifically how to correct the yoga pose of the user. The instructions may be pre-recorded for each pose or may be generated on the fly, e.g., by an artificial intelligence (AI) or machine learning (ML).

In some embodiments, the electronics unit 906 may control one or more of the scent dispensers 908, the audio speaker 910, and/or the display device 912 to output one or more stimuli for a yoga session. For example, the audio speaker 910 may be configured to output audio stimuli such as soothing or restorative music or sounds, instructions or other commentary from an instructor of a yoga session such as instructions or commentary relating to breath awareness, yoga poses, pranayama, meditation, led relaxation, and/or other practices.

Each of the scent dispensers 908 may be configured to output olfactory stimuli as part of a yoga session. Similar to other scent dispensers herein, each of the scent dispensers 908 may include a diffuser coupled to one or more scent cartridges such as those described elsewhere herein.

The display device 912 may be configured to output visual stimuli as part of a yoga session. For example, the display device 912 may be configured to output calming or soothing images or video (e.g., of nature scenes, etc.), images or video of an instructor directing the user in the yoga session, or visual feedback showing the user how to correct a yoga pose as described above. In the example of FIG. 9, the display device 912 includes a projector that may project images or video onto a nearby wall or other surface but may more generally include any suitable display device.

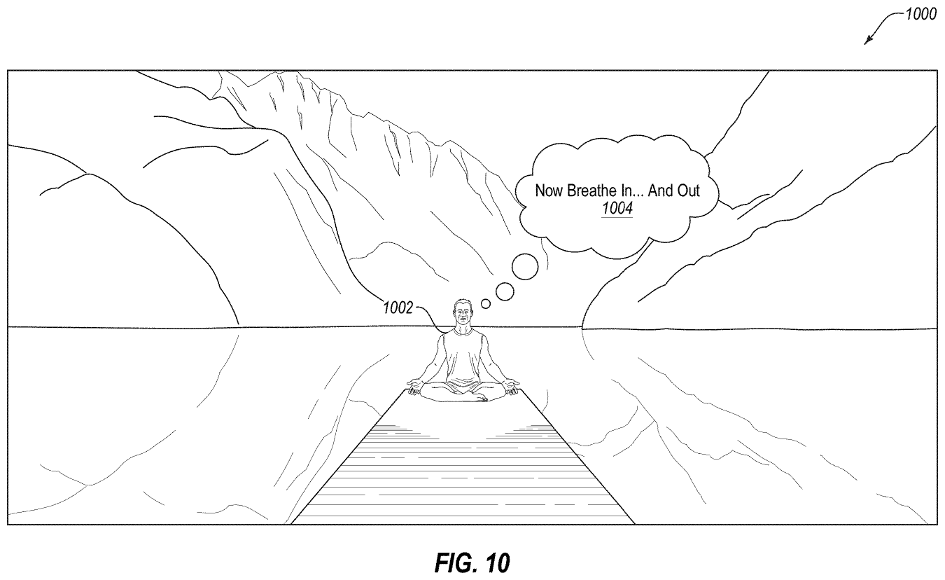

FIG. 10 illustrates a frame 1000 of a video of a video mental health program. The video mental health program in the example of FIG. 10 is for a mindfulness session. The video mental health program may include video of an instructor 1002 performing and directing a mindfulness session. The video mental health program may include output control commands to control one or more output devices of or associated with a wellness device that executes the video mental health program. As part of the mindfulness session, the instructor 1002 may direct the user to, e.g., take various actions. For example, the instructor 1002 may direct the user with respect to a breathing technique. Example instructions of the instructor 1002 with respect to the breathing technique are depicted in FIG. 10 in a speech bubble 1004 for illustrative purposes. It is understood with the benefit of the present disclosure, however, that instructions or commentary of an instructor in video programs as described herein may be provided to the user as video and/or audio of the instructor speaking the instructions or commentary.

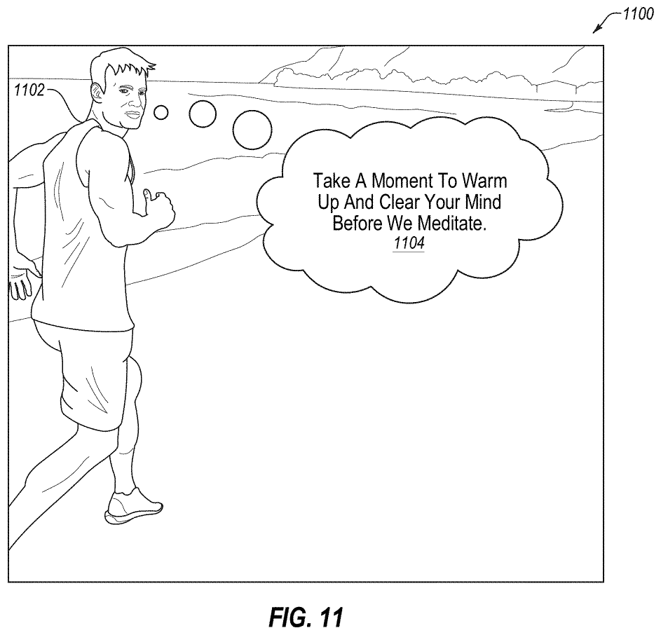

FIG. 11 illustrates a frame 1100 of a video of a video program that may include both a workout and a mental health improvement session. The video includes an instructor 1102 performing and directing a workout combined with a mindfulness session. The video program may include exercise machine control commands to control one or more movable members of an exercise machine or other wellness device that executes the video program and output control commands to control one or more output devices of or associated with the exercise machine. As part of the combined workout and mindfulness session, the instructor 1102 may direct the user to, e.g., take various actions. For example, the instructor 1102 may direct the user to warm up on the exercise machine, e.g., by jogging in this example, followed by meditation guided by the instructor 1102. Example instructions of the instructor 1102 with respect to the warm up are depicted in FIG. 11 in a speech bubble 1104 for illustrative purposes. It is understood with the benefit of the present disclosure, however, that instructions or commentary of an instructor in video programs as described herein may be provided to the user as video and/or audio of the instructor speaking the instructions or commentary.

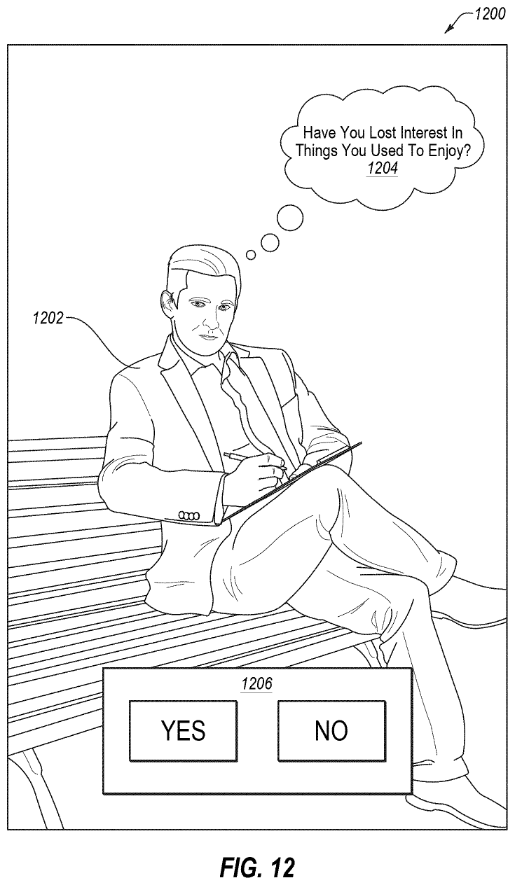

FIG. 12 illustrates a frame 1200 of a video of another video mental health program. The video mental health program in the example of FIG. 12 is for a therapy session. The video mental health program may include video of an instructor 1202, e.g., a therapist in this example, directing a therapy session. The video mental health program may include output control commands to control one or more output devices of or associated with a wellness device that executes the video mental health program. As part of the therapy session, the instructor 1202 may ask the user one or more questions that relate to a mental health of the user and/or may provide advice or counseling. An example question of the instructor 1202 is depicted in FIG. 12 in a speech bubble 1204 for illustrative purposes. It is understood with the benefit of the present disclosure, however, that instructions or commentary of an instructor in video programs as described herein may be provided to the user as video and/or audio of the instructor speaking the instructions or commentary.

In some embodiments, a user interface 1206 may be displayed in or over the video to accept user input in response to the questions. In the example of FIG. 12, the user interface 1206 is a graphical user interface in which the user can select an answer to the question. In some embodiments, the video program may be adapted according to the user's responses to the questions. For example, the questions asked by the instructor 1202 and/or advice or counsel provided by the instructor 1202 may follow a script that depends on the answers to the questions. In some embodiments, if the user answers “Yes” to a first question, the instructor 1202 may follow up the first question with a second question or with first advice or counsel; if the user answers “No” to the first question, the instructor 1202 may follow up the first question with a third question or second advice or counsel. The video program may be a live video program in which the instructor 1202 can adapt the video program or follow the script in real time. The video program may be a pre-recorded video program that includes multiple segments or branches for the various questions, advice, or counsel; after receiving the user's response to any given question, an appropriate next segment or branch may be provided to the user. Alternatively or additionally, video and/or audio of the instructor 1202 asking the questions or providing the advice or counsel may be generated on the fly, e.g., by a AI and/or ML.

Questions relating to mental health of the user may be presented to the user as part of a therapy session such as described with respect to FIG. 12, as part of other video mental health programs, or as part of video workout programs. Responses to such questions and/or other psychological parameters of the user may be collected by a user interface such as the user interface 1206 of FIG. 12 and/or in other suitable manner. Aspects of video programs or an environment of a user according to embodiments herein may be controlled based on such psychological parameters of the user and/or based on biological parameters of the user to influence the mental state of the user. For example, if during a workout or mental health improvement session the user responds to a question about mental health indicating that the user is depressed or is tending towards depression, a sun lamp may be turned on in a vicinity of the user or a video displayed to the user as part of a video program of the workout or mental health improvement session may controlled to show a path followed as part of the video emerging from a tunnel or area of relatively more darkness (e.g., a heavily forested trail) into an area with more sunlight. Video programs may be adapted by switching between different pre-recorded segments or branches and/or by generating new images or video on the fly, e.g., using a game engine.

Alternatively or additionally, workouts, mental health improvement sessions, or the like may be recommended to the user based on the user's psychological parameters and/or biological parameters. Biological parameters of the user may include biodata of the user, such as the user's heart rate, brain waves, respiratory rate, palm perspiration amount, pupil dilation amount, pupil dilation speed, sleep duration, or the like. Biological parameters of the user may alternatively or additionally include physical movement data of the user with respect to one or more prior workouts or mental health improvement sessions, target calorie burn, and/or other biological parameters. Additional details regarding recommending workouts based on one or more of the foregoing parameters are disclosed in U.S. Pre-Grant Publication No. 2018/0085630 A1 published on Mar. 29, 2018 (hereinafter the '630 publication), which is incorporated herein by reference in its entirety for all that it discloses. The methods disclosed in the '630 publication may be modified to make workout recommendations and/or mental health improvement session recommendations based on the same or different parameters disclosed therein and/or based on psychological parameters of the user as disclosed herein.

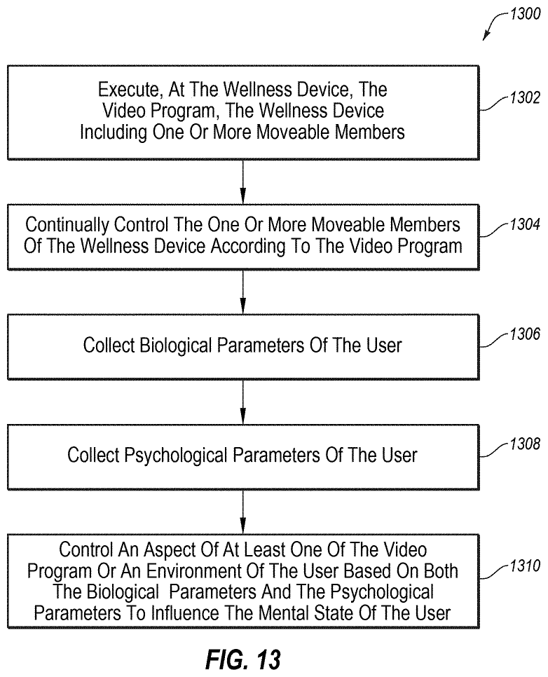

FIG. 13 illustrates a flowchart of an example method 1300 to influence mental state of a user of a wellness device with a video program. The method 1300 may be performed, in some embodiments, by one or more applications, devices, or systems, such as by the wellness devices, sensors, local servers, remote servers, or some combination thereof, and/or other applications, devices, or systems herein. In these and other embodiments, the method 1300 may be performed by one or more processors based on one or more computer-readable instructions stored on one or more non-transitory computer-readable media. The method 1300 will now be described in connection with FIGS. 1, 2, and 6A-6B.

The method 1300 may include, at action 1302, executing, at the wellness device, the video program, the wellness device including one or more moveable members. For example, the video program may be executed at the wellness devices 120a-120d of FIG. 1, the wellness device 200 of FIG. 2, and/or the immersive mental health device 600 of FIGS. 6A and 6B. Executing the video program at the wellness device may include guiding the user through a workout or a mental health improvement session.

The method 1300 may include, at action 1304, continually controlling the one or more moveable members of the wellness device according to the video program. For example, the one or more movable members may be controlled by one or more exercise machine control commands. The exercise machine control commands may be encoded in a closed caption stream of a video of the video program. In some embodiments, continually controlling the one or more moveable members at action 1304 may include continually controlling one or more of the running belt 126a, the running deck 126b, the adjustable chair 126c, or other moveable member(s) of the wellness devices 120a-120d of FIG. 1.

The method 1300 may include, at action 1306, collecting biological parameters of the user. In some embodiments, the biological parameters may be measured by one or more sensors, such as the heart rate watch 111a or the heart rate strap 111b, and collected from the sensor(s) by the wellness devices 120a-120d or other wellness devices herein. Biological parameters of the user may include biodata of the user, such as the user's heart rate, respiratory rate, palm perspiration amount, pupil dilation amount, pupil dilation speed, sleep duration, or the like, physical movement data of the user with respect to one or more prior workouts or mental health improvement sessions, target calorie burn, and/or other biological parameters.

The method 1300 may include, at action 1308, collecting psychological parameters of the user. Action 1308 may include collecting responses of the user to questions relating to mental health of the user. In this and other embodiments, the method 1300 may further include presenting the question relating to the mental health of the user to the user. For example, as described with respect to FIG. 12, an instructor such as the instructor 1202 may ask the user one or more questions relating to the mental health of the user, and the user may respond to the questions with a user interface such as the user interface 1206. Each of the questions presented to and/or answered by the user that relate to the mental health of the user may specifically relate to at least one of: a mental health history of the user, a mental health history of the user's family, sleep habits, sleep changes, mood, mood changes, anxiety, depression, stress, confusion, self-esteem, apathy, suicidal thoughts, or other aspects of mental health of the user.

The method 1300 may include, at action 1310, controlling an aspect of at least one of the video program or an environment of the user based on both the biological parameters and the psychological parameters to influence the mental state of the user. In some embodiments, the method 1300 may further include determining, by at least one of an AI or ML, the aspect to be controlled based on both the biological parameters and the psychological parameters.

The collecting of the biological parameters and/or the psychological parameters may occur before the video program begins, e.g., during a prior video program, at a beginning of the video program, or during the video program.

In some embodiments, the video program may be a first or current video program and the method 1300 may further include recommending, based on both the biological parameters and the psychological parameters, another video program to the user. For example, the other video program may have a difficulty level or intended physiological effect determined based at least on the user's biological parameters and a content or intended psychological effect determined based on the user's psychological parameters.

In some embodiments, the video program includes a video that is continuously displayed to the user during the video program and the controlling of the aspect at action 1310 includes controlling content of the video of the video program. Alternatively or additionally, controlling of the aspect at action 1310 may include controlling an output of a sun lamp in the environment of the user. In some embodiments, controlling of the aspect at action 1310 may include controlling both a first aspect of the video program and a second aspect of the environment (e.g., in the form of one or more stimuli) of the user in coordination. For example, a video of the video workout program may be controlled to follow a path out of a tunnel into sunlight while a sun lamp may be controlled in coordination to turn on and increase in brightness as the video follows the path out of the tunnel into the sunlight.

In some embodiments, the wellness device includes at least one of: an adjustable chair, a haptic device, a display device, a scent dispenser, a heater element, a cooler element, a compression member, a humidity control element, a speaker, a fan, or a light. In this and other embodiments, the controlling of the aspect at action 1310 may include controlling at least one of: recline or tilt of the adjustable chair; vibrational movement of the haptic device; applied compression by the compression member; dispensing of scent from the scent dispenser; at least one of ambient temperature, humidity, or airflow via at least one of the heater element, the cooler element, the fan, the light, or the humidity control element; light from at least one of the light or the display device; audio content from the speaker; or video content from the display device.

An implementation of the method 1300 to influence mental state of a user of an immersive mental health device with a video mental health program will now be described. The immersive mental health device may include the immersive mental health device 120d of FIG. 1, the wellness device 200 of FIG. 2, the immersive mental health device 600 of FIGS. 6A-6B, or other immersive mental health device.

In this implementation of the method 1300, the action 1302 may include executing, at the immersive mental health device, the video mental health program, the immersive mental health device including one or more moveable members. The action 1304 may include continually controlling the one or more moveable members of the immersive mental health device according to the video mental health program. The actions 1306 and 1308 may be the same, e.g., collecting biological parameters of the user at action 1306 and collecting psychological parameters of the user of action 1308. The action 1310 may include controlling an aspect of at least one of the video mental health program or an environment of the user based on both the biological parameters and the psychological parameters to influence the mental state of the user.

An implementation of the method 1300 to influence mental state of a user of an exercise machine with a video workout program will now be described. The exercise machine may include the treadmill 120a of FIG. 1, the wellness device 200 of FIG. 2, or other exercise machine.

In this implementation of the method 1300, the action 1302 may include executing, at the exercise machine, the video workout program, the exercise machine including one or more moveable members. The action 1304 may include continually controlling the one or more moveable members of the exercise machine according to the video workout program. The actions 1306 and 1308 may be the same, e.g., collecting biological parameters of the user at action 1306 and collecting psychological parameters of the user of action 1308. The action 1310 may include controlling an aspect of at least one of the video workout program or an environment of the user based on both the biological parameters and the psychological parameters to influence the mental state of the user.

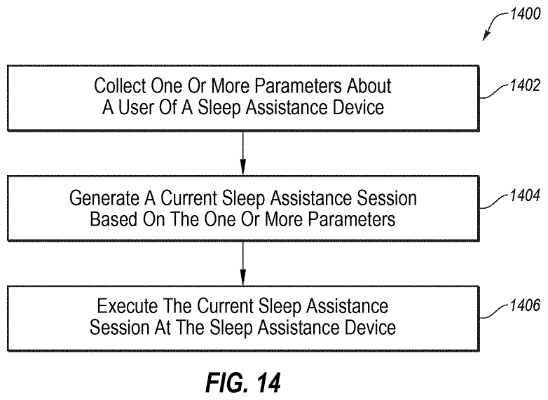

FIG. 14 illustrates a flowchart of an example method 1400 to help a user of a sleep assistance device sleep. The method 1400 may be performed, in some embodiments, by one or more applications, devices, or systems, such as by the wellness devices, sensors, local servers, remote servers, or some combination thereof, and/or other applications, devices, or systems herein. In these and other embodiments, the method 1400 may be performed by one or more processors based on one or more computer-readable instructions stored on one or more non-transitory computer-readable media. In an example implementation, the method 1400 is performed at or by a sleep assistance device with a main body, a processing unit, an audio speaker, a light source, a scent dispenser, a display device, and a computer storage. The method 1400 will now be described in connection with FIGS. 1, 3, and 7A-7H.

The method 1400 may include, at action 1402, collecting one or more parameters about a user of the sleep assistance device. The one or more parameters may include at least one of one or more biological parameters or one or more psychological parameters. The biological parameters may be measured by one or more sensors, such as the heart rate watch 111a or the heart rate strap 111b, and collected from the sensor(s) by the sleep assistance device 120b of FIG. 1, the sleep assistance device 300 of FIG. 3, the sleep assistance devices 700a-700e of FIGS. 7A-7H, or other sleep assistance devices herein. Biological parameters of the user may include biodata of the user, such as the user's heart rate, respiratory rate, palm perspiration amount, pupil dilation amount, pupil dilation speed, sleep duration, or the like, physical movement data of the user with respect to one or more prior workouts or mental health improvement sessions, target calorie burn, and/or other biological parameters. Collecting psychological parameters of the user may include collecting responses of the user to questions relating to mental health of the user, examples of which are described elsewhere herein.

In some embodiments, the collecting of the one or more parameters at action 1402 or at other actions in other methods herein may include collecting the one or more parameters from an online profile of the user. For example, information regarding workouts or mental health improvement sessions completed by the user using one or more of the wellness devices herein may be uploaded by the wellness device or other device to an online fitness profile, wellness profile, or other social media profile of the user. The information may include biological parameters, psychological parameters, or other parameters derived or collected in association with administration of a video workout program or video mental health program to the user at an exercise machine (such as the treadmill 120a) or an immersive mental health device (such as the immersive mental health device 120d, 600).

The method 1400 may include, at action 1404, generating a current sleep assistance session based on the one or more parameters, the current sleep assistance session configured to assist the user to reach and remain in a sleep state. As indicated previously, each sleep assistance session may include one or more aural, visual, olfactory, and/or tactile stimuli that are collectively configured to assist a user to reach and remain in a sleep state and/or to wake from a sleep state. Aspects of the sleep assistance session may be determined, e.g., by AI and/or ML, to assist the user to reach and remain in a sleep state while taking into account the user's mental state and/or physical state as indicated by the parameters of the user. For example, if the parameters indicate the user is experiencing a particular physical and/or mental state (e.g., anxious or stressed), the sleep assistance session may be generated by selecting one or more aural, visual, olfactory, and/or tactile stimuli known or suspected to help the general population fall asleep under the same physical and/or mental state or known or suspected to help the general population address the same physical and/or mental state before falling asleep. Alternatively or additionally, content or other details of one or more prior sleep assistance sessions and parameters of the user before and after the prior sleep assistance sessions may be stored, e.g., in a profile of the user, and the sleep assistance session may be generated by selecting one or more aural, visual, olfactory, and/or tactile stimuli known or suspected to help the specific user fall asleep under the same physical and/or mental state or known or suspected to help the specific user address the same physical and/or mental state before falling asleep.

The method 1400 may include, at action 1406, executing the current sleep assistance session at the sleep assistance device, including coordinating operation of the audio speaker, the light source, the scent dispenser, and the display device to output coordinated aural, visual, and olfactory stimuli to the user that are configured to assist the user to reach and remain in the sleep state. For example, the action 1406 may include the processing unit 302 of FIG. 3 coordinating operation of the audio speaker 310, 704a-704e, the light source 308, 706a-706e, the scent dispenser 306, 708a-708e, and the display device 312, 710a-710e of the sleep assistance device 300, 700a-700e to output coordinated aural, visual, and olfactory stimuli to the user. Executing the current sleep assistance session may include operating at least one of the light source or the display device to output time-varying light that mimics time-varying light from a sunset.

In some embodiments, the processing unit of the sleep assistance device is communicatively coupled to one or more sensors in operational proximity to the user as the user sleeps. The collecting of the one or more parameters at action 1402 may include collecting from the one or more sensors one or more measurements of the user generated as the user sleeps during a prior sleep session of the user. In this and other embodiments, the generating of the current sleep assistance session may include modifying a prior sleep assistance session based on a reaction of the user to the prior sleep assistance session reflected in the one or more measurements of the user generated as the user sleeps during the prior sleep session of the user.

In some embodiments, the method 1400 may further include generating a current wake assistance session at the sleep assistance device based on the one or more parameters. The current wake assistance session may be configured to assist the user to awake from the sleep state. The method 1400 may further include executing the current wake assistance session at the sleep assistance device, including coordinating operation of the audio speaker, the light source, the scent dispenser, and the display device to output coordinated aural, visual, and olfactory stimuli to the user that are configured to wake the user from the sleep state. Executing the current wake assistance session may include operating at least one of the light source or the display device to output time-varying light that mimics time-varying light from a sunrise.

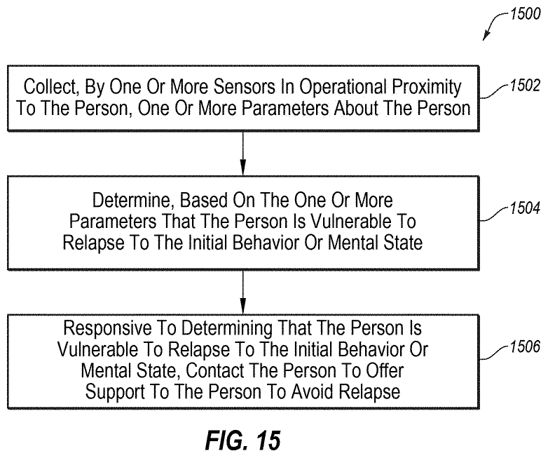

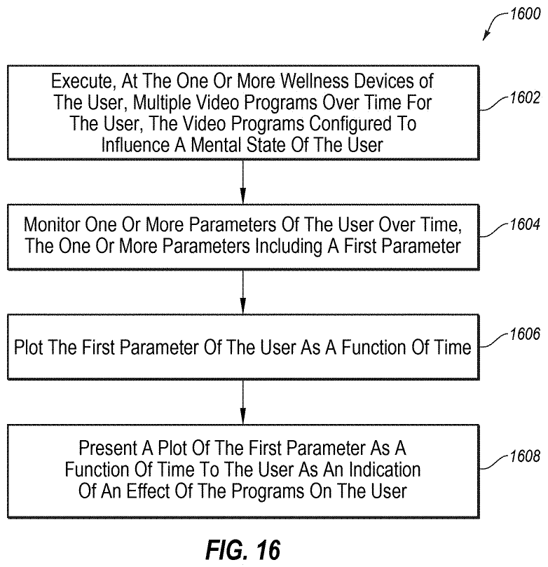

Some embodiments herein may utilize concepts of accountability, assessment, and/or progress to aid wellness device users and/or other persons to improve their physical and/or mental health. Various examples are described with respect to FIGS. 15 and 16.

FIG. 15 illustrates a flowchart of an example method 1500 to make a person accountable to change behavior or mental state from an initial behavior or mental state to a target behavior or mental state. The method 1500 may be performed, in some embodiments, by one or more applications, devices, or systems, such as by the wellness devices, sensors, local servers, remote servers, or some combination thereof, and/or other applications, devices, or systems herein. In these and other embodiments, the method 1500 may be performed by one or more processors based on one or more computer-readable instructions stored on one or more non-transitory computer-readable media. In an example implementation, the method 1500 is performed at or by the local server 116 or the remote server 112.

The method 1500 may include, at action 1502, collecting, by one or more sensors in operational proximity to the person, one or more parameters about the person. For example, the action 1502 may include collecting a parameter by a sensor of a personal electronic device borne by the person, such as a smartphone, a wearable electronic device (e.g., smart watch), or other personal electronic device.

Some embodiments herein allow or encourage the person to keep a digital journal. Keeping a journal, e.g., making one or more entries in the journal, may be therapeutic for the person. The journal may be available online and/or may be accessed by the person through an online fitness platform with which the person has a fitness account and/or a fitness profile, an online wellness platform with which the person has a wellness account and/or a wellness profile, a social media platform with which the person has a social medial account and/or a social medial profile, or other platform, system, or website. In this and other embodiments, the action 1502 may include collecting a parameter by natural language processing of a journal entry of the person entered into the digital journal by the person. The journal entry or information derived therefrom may indicate a mental state of the person at the time of the journal entry.

In some embodiments, the action 1502 may include collecting a parameter by an exercise machine used by the person to perform a workout or a mental health improvement session. The parameter may include a biological parameter, a psychological parameter, or other parameter collected by the exercise machine. In some embodiments, the action 1502 may include collecting a parameter by a digital device used by the person to play a game. The parameter may include an identification of the game, a time of the person to complete the game, an indication of whether the person completed the game or terminated the game prior to completion, or other parameter about the game or the person's play of the game. If the person is taking longer than usually to complete the game or is terminating the game prior to completion, the person may be stressed or anxious or in some other mental state. In some embodiments, the action 1502 may include collecting a parameter by an immersive mental health device used by the person to perform a mental health improvement session. In some embodiments, the action 1502 may include collecting a parameter from the fitness profile, the wellness profile, or the social medial profile of the person.

The method 1500 may include, at action 1504, determining based on the one or more parameters that the person is vulnerable to relapse to the initial behavior or mental state. The determination at action 1504 may use AI/ML to identify if and when the person is vulnerable to relapse based on the one or more parameters. For example, a training set of parameters of persons that have relapsed may be used to generate a population-based relapse model and the AI/ML may apply the population-based relapse model to the one or more parameters of the person to determine if the person is vulnerable to relapse. In some embodiments, persons that relapse may experience the same or similar changes in one or more parameters leading up to relapse. If the one or more parameters of the person appear to be following the same or similar changes as those of the persons that relapsed, it may be determined at action 1504 that the person is vulnerable to relapse.

Alternatively or additionally, a person-specific relapse model may be generated by the AI/ML and applied in the same or similar manner as the population-based relapse model to determine whether the person is vulnerable to relapse at action 1504. The person-specific relapse model may be generated from one or more parameters of the person leading up to a known relapse by the person. For example, the person after relapse may access their fitness profile, wellness profile, social media profile, or the like to voluntarily identify that the person had a relapse and the time of the relapse and the AI/ML may generate the person-specific model from the one or more parameters of the person at least leading up to the relapse time, e.g., for the 30 minutes prior to the relapse time.

Alternatively or additionally, the method 1500 may further include determining that the person relapsed to the initial behavior, determining a relapse time at which the person relapsed to the initial behavior, and analyzing one or more previous parameters of the person captured during an interval of a predetermined duration that begins prior to the relapse time and terminates at the relapse time to identify one or more indications in the one or more previous parameters of the person that the person was vulnerable to the relapse. In this and other embodiments, the determining based on the one or more parameters that the person is vulnerable to relapse to the initial behavior at the action 1504 may include identifying one or more current indications in the one or more parameters that are similar or identical to the one or more indications identified in the one or more previous parameters.

The method 1500 may include, at action 1506, and responsive to determining that the person is vulnerable to relapse to the initial behavior or mental state, contacting the person to offer support to the person to avoid relapse. In some embodiments, the contacting of the person may include directly contacting the person via e-mail, text message, voice call, voice message, video call, video message, or the like. The e-mail, text message, voice call, voice message, video call, video message, or the like may include a computer-generated deepfake representation of someone known to the person. In some embodiments, the person is a first person and the contacting of the first person includes indirectly contacting the first person by arranging for a second person to directly contact the first person.

In some embodiments, the initial behavior or mental state may include at least one of: smoking, vaping, being sedentary, overconsuming food, consuming or overconsuming alcohol, consuming or overconsuming drugs, insomnia, anxiety, or depression. In this and other embodiments, the target behavior or mental state may include at least one of: not smoking, not vaping, exercising, not overconsuming food, not consuming or overconsuming alcohol, not consuming or overconsuming drugs, sleeping, or mental equilibrium.

FIG. 16 illustrates a flowchart of an example method 1600 to improve a mental health of a user of one or more wellness devices. The wellness devices may include any of the wellness devices herein. The method 1600 may be performed, in some embodiments, by one or more applications, devices, or systems, such as by the wellness devices, sensors, local servers, remote servers, or some combination thereof, and/or other applications, devices, or systems herein. In these and other embodiments, the method 1500 may be performed by one or more processors based on one or more computer-readable instructions stored on one or more non-transitory computer-readable media. In an example implementation, the method 1500 is performed at or by any of the wellness devices herein alone or in combination with the local server 116, the remote server 112, or other computing device.

The method 1600 may include, at action 1602, executing, at the one or more wellness devices of the user, multiple video programs over time for the user, the video programs configured to influence a mental state of the user. The video programs may include one or more video workout programs and/or video mental health programs.

The method 1600 may include, at action 1604, monitoring one or more parameters of the user over time, the one or more parameters including a first parameter. The one or more parameters may include biological parameters, psychological parameters, or other parameters. Alternatively or additionally, the monitoring of the one or more parameters of the user over time at the action 1604 may include collecting at least one of passive feedback from the user or active feedback from the user. In general, parameters or other feedback collected from the user may be considered passive feedback if collection thereof does not require or involve any mental thought on the part of the user or active feedback if collection thereof requires or involves mental thought on the part of the user. In some embodiments, passive feedback may include the user's heart rate, respiratory rate, palm perspiration amount, pupil dilation amount or speed, sleep duration, or other parameter that may be measured and provided by a sensor on or in operational proximity to the user without thought by the user. In some embodiments, active feedback may include responses of the user to questions related to mental health of the user, a journal entry of the user, or other feedback from the user that requires or involves mental thought on the part of the user.

The method 1600 may include, at action 1606, plotting the first parameter of the user as a function of time.

The method 1600 may include, at action 1608, presenting a plot of the first parameter as a function of time to the user as an indication of an effect of the video programs on the user. Presenting the plot to the user may convey to the user any progress that the user is making, which may encourage the user to continue using the video programs. For example, if the user is stressed or anxious regularly, the user may have an elevated resting heart rate as a result of the user's regularly stressed/anxious mental state. Such a mental state may be detrimental to the wellbeing of the user. The video programs executed at the one or more wellness devices may be configured generally to influence the mental state of the user, and specifically to help reduce the user's stress and/or anxiety in this example. By monitoring the user's heart rate at the action 1604, plotting the user's heart rate as a function of time at the action 1606, and presenting a plot of the user's heart rate as a function of time to the user at the action 1608, the user may be able to see whether the video programs are helping reduce the user's stress and/or anxiety as may be indicated by a decline over time in the user's resting heart rate. If the plot presented to the user indicates the video programs are having their intended effect (whether it be reducing the user's stress and/or anxiety or other intended effect), the user may be encouraged to continue using the video programs. If the plot presented to the user indicates the video programs are not having their intended effect, the user may take other steps in pursuit of the intended effect, e.g., increasing or decreasing an amount of time per day or week spent using the video programs, using different video programs than the user has been using, adding or removing video programs for different types of workouts and/or mental health improvement sessions than the user has been doing, or the like.

In some embodiments, the method 1600 may further include recommending one or more of the video programs to the user based on the one or more parameters, e.g., as described with respect to FIG. 13.

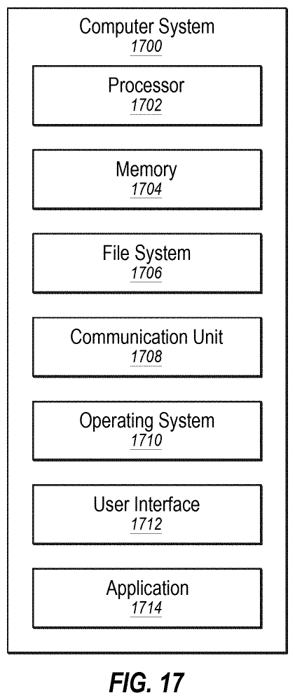

FIG. 17 illustrates an example computer system 1700 that may be employed in performing or controlling performance of one or more of the methods or actions herein. In some embodiments, the computer system 1700 may be part of any of the systems or devices described in this disclosure. For example, the computer system 1700 may be part of any of the video cameras 106a-106c, the computer 114, the remote server 112, the game engine 115, the local server 116, the wellness devices 120a-120d, the console 122, or the tablet 124 of FIG. 1.

The computer system 1700 may include a processor 1702, a memory 1704, a file system 1706, a communication unit 1708, an operating system 1710, a user interface 1712, and an application 1714, which all may be communicatively coupled. In some embodiments, the computer system may be, for example, a desktop computer, a client computer, a server computer, a mobile phone, a laptop computer, a smartphone, a smartwatch, a tablet computer, a portable music player, an exercise machine console, a video camera, or any other computer system.

Generally, the processor 1702 may include any suitable special-purpose or general-purpose computer, computing entity, or processing device including various computer hardware or software applications and may be configured to execute instructions stored on any applicable computer-readable storage media. For example, the processor 1702 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data, or any combination thereof. In some embodiments, the processor 1702 may interpret and/or execute program instructions and/or process data stored in the memory 1704 and/or the file system 1706. In some embodiments, the processor 1702 may fetch program instructions from the file system 1706 and load the program instructions into the memory 1704. After the program instructions are loaded into the memory 1704, the processor 1702 may execute the program instructions. In some embodiments, the instructions may include the processor 1702 performing one or more actions of the methods 1300, 1400, 1500, 1600 of FIGS. 13-16.

The memory 1704 and the file system 1706 may include computer-readable storage media for carrying or having stored thereon computer-executable instructions or data structures. Such computer-readable storage media may be any available non-transitory media that may be accessed by a general-purpose or special-purpose computer, such as the processor 1702. By way of example, and not limitation, such computer-readable storage media may include non-transitory computer-readable storage media including Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage media which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 1702 to perform a certain operation or group of operations, such as one or more actions of the methods 1300, 1400, 1500, 1600 of FIGS. 13-16. These computer-executable instructions may be included, for example, in the operating system 1710, in one or more applications, or in some combination thereof.

The communication unit 1708 may include any component, device, system, or combination thereof configured to transmit or receive information over a network, such as the network 118 of FIG. 1. In some embodiments, the communication unit 1708 may communicate with other devices at other locations, the same location, or even other components within the same system. For example, the communication unit 1708 may include a modem, a network card (wireless or wired), an infrared communication device, a wireless communication device (such as an antenna), and/or chipset (such as a Bluetooth device, an 802.6 device (e.g., Metropolitan Area Network (MAN)), a WiFi device, a WiMax device, a cellular communication device, etc.), and/or the like. The communication unit 1708 may permit data to be exchanged with a network and/or any other devices or systems, such as those described in the present disclosure.

The operating system 1710 may be configured to manage hardware and software resources of the computer system 1700 and configured to provide common services for the computer system 1700.

The user interface 1712 may include any device configured to allow a user to interface with the computer system 1700. For example, the user interface 1712 may include a display, such as an LCD, LED, or other display, that is configured to present video, text, application user interfaces, and other data as directed by the processor 1702. The user interface 1712 may further include a mouse, a track pad, a keyboard, a touchscreen, volume controls, other buttons, a speaker, a microphone, a camera, any peripheral device, or other input or output device. The user interface 1712 may receive input from a user and provide the input to the processor 1702. Similarly, the user interface 1712 may present output to a user.

The application 1714 may be one or more computer-readable instructions stored on one or more non-transitory computer-readable media, such as the memory 1704 or the file system 1706, that, when executed by the processor 1702, is configured to perform one or more actions of the methods 1300, 1400, 1500, 1600 of FIGS. 13-16. In some embodiments, the application 1714 may be part of the operating system 1710 or may be part of an application of the computer system 1700, or may be some combination thereof. In some embodiments, the application 1714 may include a machine learning model. In general, the machine learning model may be trained based on sample data, known as training data, in order to make predictions or decisions without being explicitly programmed to do so. The machine learning model may employ machine learning algorithms, and may be supervised or unsupervised. The machine learning model may be trained over time to become more and more accurate. The machine learning model may be trained, for example, using a Decision Tree, Naive Bayes Classifier, K-Nearest Neighbors, Support Vector Machines, or Artificial Neural Networks. The machine learning model may be employed in any of the methods herein to perform actions with increasing effectiveness and accuracy over time, as the machine learning model learns and is periodically retrained to make more accurate predictions or decisions. For example, any of the actions 1304, 1310, 1404, 1406, 1504, 1506, and 1602, or any other action, may be performed by the machine learning model in order to perform these actions with increasing effectiveness and accuracy over time as the machine learning model learns.

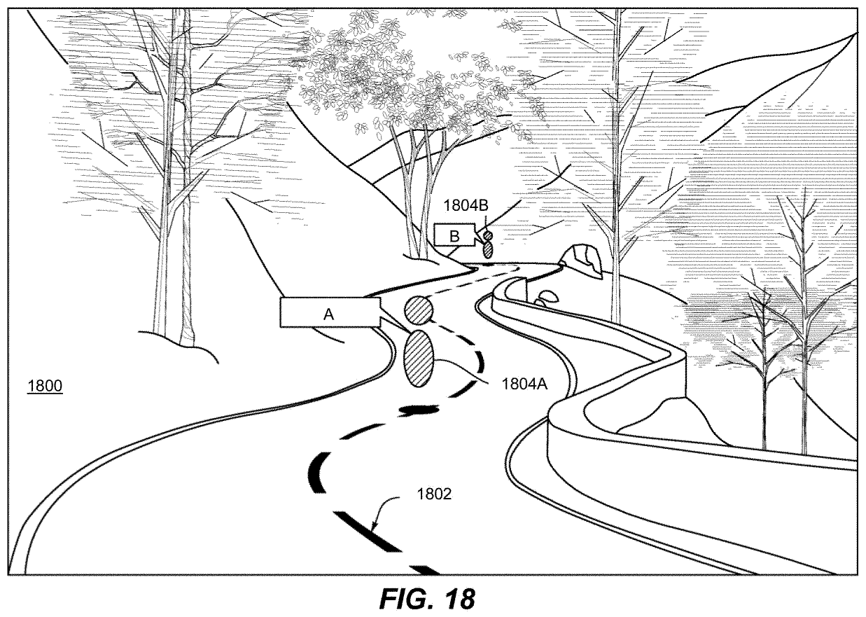

FIG. 18 illustrates an example view of an example virtual environment 1800. In some embodiments the virtual environment may be rendered in UNREAL ENGINE. The virtual environment 1800 may be configured to provide an engaging workout experience as a user travels through the virtual environment 1800 in accordance with the actions of the user on exercise equipment. For example, the user may use a treadmill and the virtual environment 1800 may depict a road. The depiction of the road may change as the user runs on the treadmill in such a way as to simulate movement through the virtual environment at the same speed as the operation of the treadmill. A workout associated with a virtual environment 1800 may provide a more engaging experience for the user than a workout associated with a real-world environment or a workout not associated with any environment. Use of a virtual environment 1800 may provide the user with greater options for workout experiences and may result in more effective and more engaging workouts.

The virtual environment 1800 may be displayed on a display of exercise equipment, a display of a computing device, or a display of a virtual reality or augmented reality headset. A view of the virtual environment 1800 may update on the display in order to simulate movement through the virtual environment 1800. For ease of discussion, the simulation of movement of a user through the virtual environment 1800 will simply be referred to as movement of the user through the virtual environment 1800 (e.g. “the user moves through the virtual environment 1800”).

The virtual environment 1800 includes a path 1802 along which the user moves. The path 1802 may be marked in the virtual environment 1800 by lines or markers. The path 1802 may run along a three-dimensional surface of the virtual environment 1800. In some embodiments the path 1802 may rise above the surface of the virtual environment 1800 or pierce the surface of the virtual environment 1800. The path 1802 may have an incline associated with the incline of the surface of the virtual environment 1800.

The virtual environment may include one or more images 1804A and 1804B referred to collectively as images 1804. The images 1804 may travel along the path 1802. The images may be labeled. For example, image 1804A may be labeled “A” and image 1804B may be labeled “B.” The images 1804 may travel at a fixed speed, at a speed relative to the speed of the user, or at a speed dependent upon the incline of the path 1802. The images 1804 may serve as motivation to the user as the user travels along the path 1802. The images 1804 may appear as traveling along the path 1802 in the same manner as the user. For example, if the user is running on a treadmill, the images 1804 may appear to be running while if the user is on a stationary bike, the images 1804 may appear to be cycling. In some embodiments the images 1804 may represent the movement of a trainer or other users along the path 1802. For example, image 1804A may represent a trainer. The trainer may travel along the path at a pace representing a predetermined workout. Alternatively, the trainer may travel along the path at a dynamic pace configured to encourage the user to improve a strength or endurance of the user. In a second example, image 1804B may represent a second user. Image 1804B may represent the progress of the second user along the path 1802 as the second user exercises on second exercise equipment and travels through the virtual environment 1800. Image 1804B may represent the progress of the second user in real-time or the recorded progress of the second user as the second user traveled through the virtual environment. Image 1804B may travel along the path 1802 according to the recorded progress of the second user such that image 1804B represents the second user starting at the same time as the user. This allows the user to race against the second user even when the user begins the workout later than the second user. In a third example, Image 1804A may represent a trainer as in the first example and Image 1804B may represent a second user as in the second example. The trainer and the second user may coexist in the virtual environment 1800. This allows the user to receive instruction and encouragement from the trainer while racing against the second user.

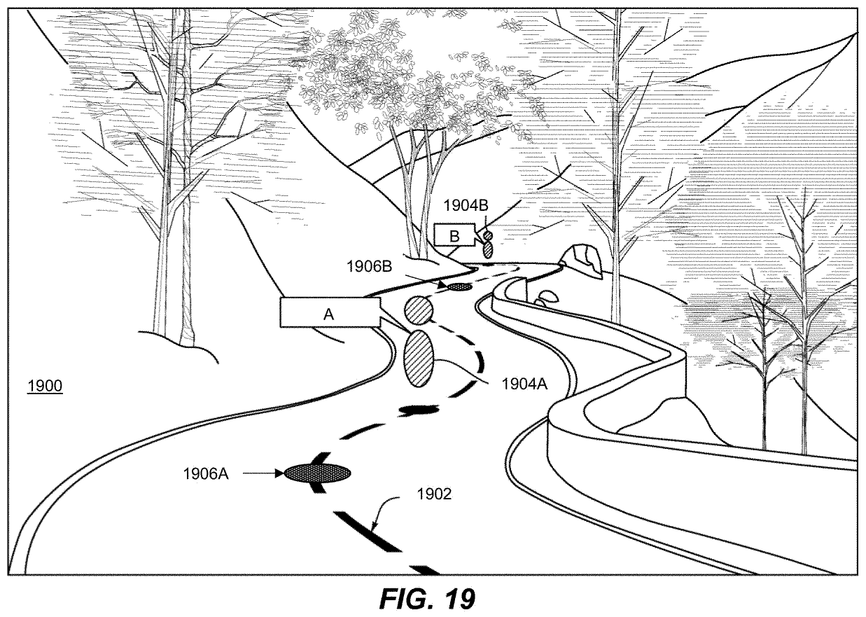

FIG. 19 illustrates an example view of an example virtual environment 1900 with control signal checkpoints 1906. The virtual environment 1900 may be similar in many respects to the virtual environment 1800. A user may travel through the virtual environment 1900 according to one or more parameters of exercise equipment. The one or more parameters of the exercise equipment may be controlled by one or more actuators. For example, a treadmill may include an actuator which controls a speed of a belt of the treadmill and an actuator which controls an incline of the belt of the treadmill. A user may travel through the virtual environment 1900 with the one or more parameters of the exercise equipment corresponding with features of a path 1902 of the virtual environment 1900. The one or more parameters of the exercise equipment may be controlled using control signals. For example, a user on a stationary bike including an actuator controlling a resistance of the stationary bike may travel through the virtual environment 1900 at a speed corresponding to a speed of the pedals of the stationary bike and with the resistance of the stationary bike corresponding to an incline of the path 1902 of the virtual environment 1900. The parameters of the exercise equipment may be updated at one or more control signal checkpoints 1906 on the path 1902 of the virtual environment 1900. In some embodiments the virtual environment 1900 updates the control signals only at the control signal checkpoints 1906. This embodiment has an advantage of reducing the frequency of updates to the control signals. The parameters of the exercise equipment may be limited in how quickly they can be adjusted. The speed of adjustments to the parameters of the exercise equipment may also be limited by how quickly the parameters can be safely adjusted. For example, a treadmill having an actuator which controls the incline of a belt of the treadmill may be capable of receiving frequent control signals, but the actuator may be limited in how quickly it can adjust the incline of the belt. Receiving control signals more rapidly than the actuator can adjust may result in inconsistent, unexpected movement of the belt. Furthermore, it may be dangerous to a user to rapidly or frequently adjust the incline of the belt. A user might lose their balance and fall, resulting in injury. Thus, restricting the adjustment of the exercise machine parameters to checkpoints has the technical advantage of giving actuators of exercise machines time to adjust the parameters of the exercise machine to create a more realistic and safer simulation of movement through the virtual environment.

The control signal checkpoints 1906 are associated with control signals corresponding to features of the path 1902 of the virtual environment 1900 and/or a workout. For example, a first control signal checkpoint 1906A may be associated with a control signal of 10 degrees incline corresponding to an incline of 10 degrees of the path 1902 at the location of the first control signal checkpoint 1906A. The control signal causes an actuator of a treadmill to incline a belt of the treadmill to 10 degrees. In a second example, the first control signal checkpoint 1906A may be associated with a control signal of 10 miles per hour corresponding to a workout dictating a speed of 10 miles per hour at a location of the first control signal checkpoint 1906A. The control signal causes an actuator of a treadmill to run a belt of the treadmill at 10 miles per hour. In a third example, the first control signal checkpoint 1906A may be associated with a control signal of 10 degrees incline and 10 miles per hour corresponding to an incline of 10 degrees of the path 1902 at the location of the first control signal checkpoint 1906A and a workout dictating a speed of 10 miles per hour at a location of the first control signal checkpoint 1906A. The control signal causes a first actuator of a treadmill to incline a belt of the treadmill to 10 degrees and a second actuator of the treadmill to run the belt at 10 miles per hour.

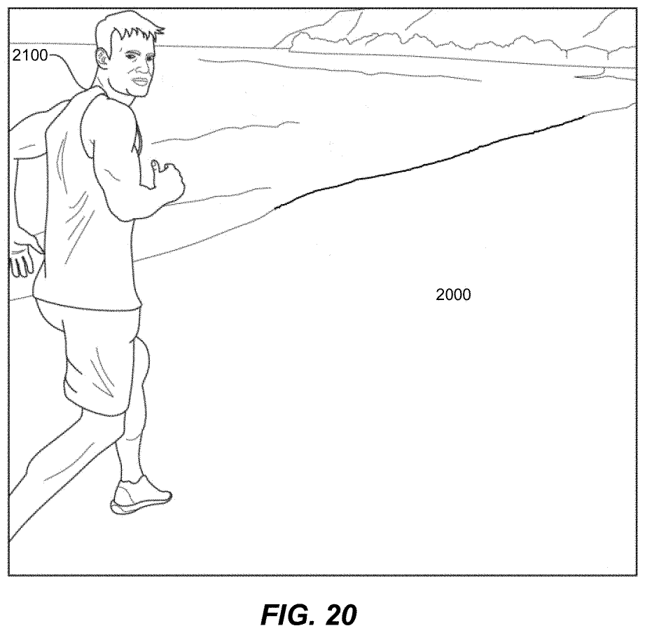

FIG. 20 illustrates an example view of trainer 2100 running in an example virtual environment 2000. The trainer 2100 may be a real person placed within the virtual environment 2000. In some embodiments the trainer 2100 may be a virtual trainer in the virtual environment 2000. The trainer 2100 may run through the virtual environment 2000 according to a workout program. The workout program may include control signals to control actuators of exercise equipment as disclosed herein according to the workout plan. Controlling the actuators of the exercise equipment according to the workout plan may allow a user to move realistically through the virtual environment 2000 with the trainer 2100. The trainer 2100 may offer instruction and/or encouragement to the user.

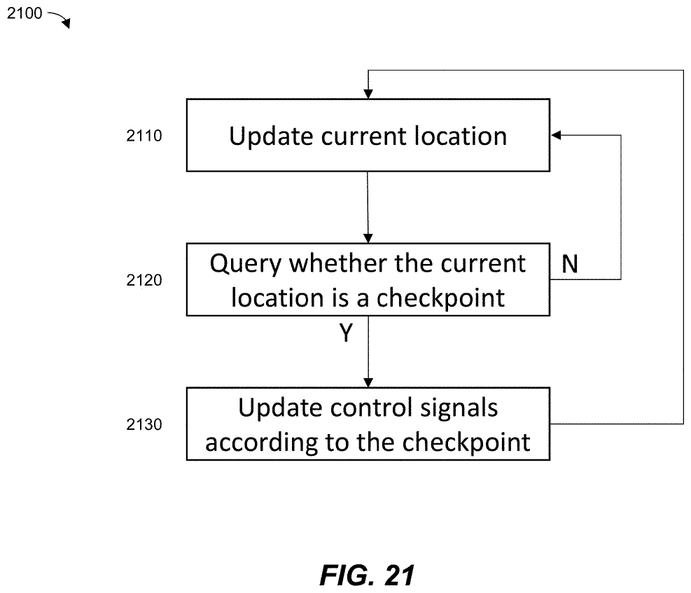

FIG. 21 illustrates a flowchart 2100 of an example method for updating control signals at checkpoints in the virtual environment 1900. Additional, fewer, or different operations may be performed in the method, depending on the embodiment. Further, the operations may be performed in the order shown, concurrently, or in a different order. At 2110 a processor of exercise equipment of a user updates a location of the user in the virtual environment 1900. At 2120 the processor queries whether the location of the user in the virtual environment 1900 is a checkpoint 1906. If the location of the user is not a checkpoint 1906 the processor updates the location of the user according to a speed of the exercise equipment without updating control signals to actuators of the exercise equipment. If the location of the user is a checkpoint 1906, the processor updates the control signals to the actuators of the exercise equipment at 2130 and then updates the location of the user according to a speed of the exercise equipment.

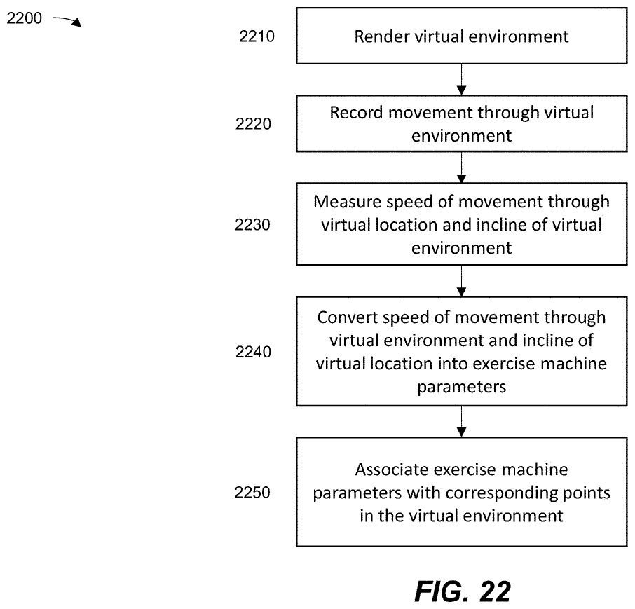

FIG. 22 illustrates a flowchart of an example method 2200 for automatically associating control signals with points in a virtual environment. Additional, fewer, or different operations may be performed in the method, depending on the embodiment. Further, the operations may be performed in the order shown, concurrently, or in a different order. At 2210 a virtual environment is rendered. At 2220 movement through the rendered virtual environment is recorded. The movement through the rendered virtual environment may be along a path of the virtual environment. At 2230 a speed of the movement through the virtual environment is recorded as well as an incline of the virtual environment under the movement through the virtual environment. At 2240 the speed of the movement and the incline of the virtual environment are converted into exercise machine parameters. For example, the speed of the movement may be converted into a speed of a belt of a treadmill and the incline of the virtual environment may be converted into an incline of the belt of the treadmill. The exercise machine parameters may correspond to the actual values of the speed of the movement and incline of the virtual environment or they may be altered according to a scaling factor. For example, a speed of 10 miles per hour of the movement may be scaled down to 7 miles per hour and an incline of 30 degrees may be scaled down to 12 degrees. At 2250 the exercise machine parameters are associated with corresponding points in the virtual environment. For example, control signal checkpoints, as disclosed herein, may be created for the virtual environment. In another example, the exercise machine parameters and/or associated control signals may be associated with timestamps. The timestamps may correspond to a time during the movement through the virtual environment when the speed of the movement and the incline of the virtual environment corresponds to the speed and incline of the exercise machine parameters. Using timestamps to associate control signals with the virtual environment has a technical advantage of separating the control signals from the video, resulting in lower computing costs. Rendering the virtual environment to extract the control signals is computationally expensive. By separating the control signals from the video, the video can be simplified to contain only the audio/visual information of the workout. The control signals will still be synchronized with the video via the timestamps. The control signals are sent to actuators of the exercise machine at the same time as they would if the virtual environment were being rendered in real-time, but the computational expense is much lower. This can result in more reliable playback of the video and execution of the control signals.

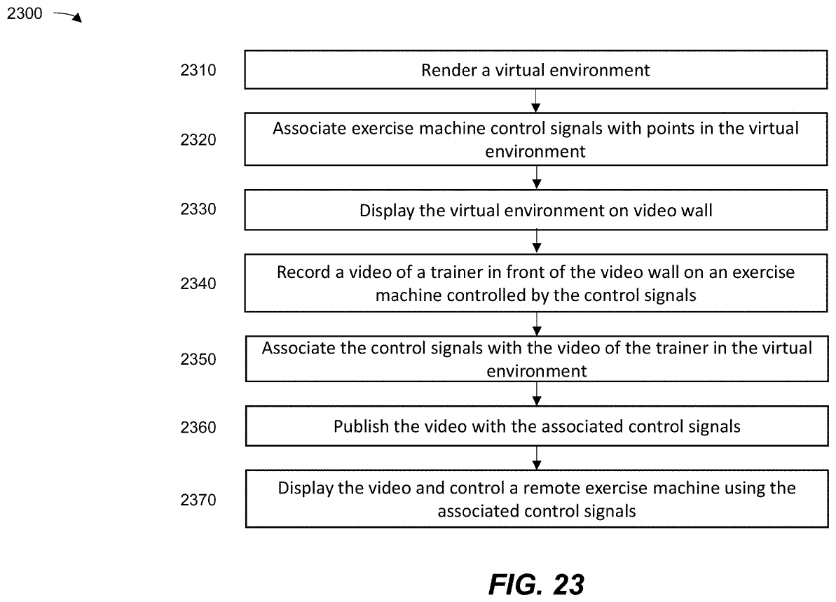

FIG. 23 illustrates a flowchart of an example method 2300 for creating and publishing a video of a trainer in a virtual environment. Additional, fewer, or different operations may be performed in the method, depending on the embodiment. Further, the operations may be performed in the order shown, concurrently, or in a different order. At 2310 the virtual environment is rendered. In some embodiments the virtual environment is rendered in UNREAL ENGINE.

At 2320 exercise machine control signals are associated with points in the virtual environment. In some embodiments the exercise machine control signals are associated with points in the virtual environment according to the method described in FIG. 22. In other embodiments, the exercise machine control signals are manually associated with points in the virtual environment. In yet other embodiments, the exercise machine control signals are associated with points in the virtual environment via timestamps that correspond to portions of a video of movement through the virtual environment. For example, a video may be captured of movement through a virtual environment and exercise machine controls may be associated with timestamps of the video to corresponding to portions of the video where the speed of the movement and the incline of the virtual environment correspond to a speed and/or incline of the exercise machine controls.

At 2330 the virtual environment is displayed on a video wall. The video wall may be an LED screen, a series of LED screens, or other type of display. In some embodiments the virtual environment may be rendered on the video wall. In other embodiments, a video of motion through the virtual environment may be displayed on the video wall.

At 2340 a video is recorded of a trainer on an exercise machine controlled by the control signals in front of the video wall displaying the virtual environment. For example, the virtual environment may be a station on the planet Mars and the exercise machine may be a treadmill. The video is then a video of a trainer running in the station on Mars with the motions of the trainer coordinated, via the exercise machine controls, with the movement of the trainer through the station on Mars. The trainer's speed matches the speed of the movement through the station and the trainer's incline matches the incline of a path through the station. This allows a real-world trainer to be filmed in a virtual location. This may provide the personal connection and encouragement of a trainer as well as the excitement and engagement of running in a virtual environment. This process also affords a technical advantage of eliminating several pre-filming and post-processing tasks involved in placing real-world people in virtual environments. Filming in front of a video wall eliminates the need to rotoscope or key the trainer into the virtual environment. Additionally, it greatly reduces the need to match the lighting on the trainer to the lighting of the virtual environment since the virtual environment is illuminating the trainer via the video wall. The trainer can also react to the virtual environment because it is displayed on the video wall, as opposed to captured separately and then keyed in. Controlling the parameter of the exercise machine using control signals associated with the virtual environment has the technical advantage of automatically synchronizing the motions of the trainer with the movement through the virtual environment. This eliminates the need to manually adjust either the speed of the exercise machine or the speed of the video to synchronize the motions of the trainer and the video. This allows for a live exercise class in a virtual environment led by a real-world trainer with greatly reduced computational cost and technical requirements. For example, a file containing control signals and associated timestamps may be loaded onto a treadmill of a trainer as well as one or more remote treadmills of remote users. A video of the trainer running in front of a video wall displaying the virtual environment may be broadcast to the one or more remote treadmills. This way, the only thing that needs to be broadcast is the video of the trainer in front of the video wall, greatly reducing the computational cost and complexity of filming a trainer in a virtual environment.

At 2350 the exercise machine controls are associated with the video of the trainer in the virtual environment. The exercise machine controls may be associated with timestamps corresponding to portions of the video when the exercise machine of the trainer was controlled by the exercise machine controls. For example, if a control signal caused a treadmill of a trainer to run at 8 miles per hour at time 1:32 of the video of the trainer running in the virtual environment, then the control signal of 8 miles per hour may be associated with the time stamp 1:32.

At 2360 the video with the associated control signals is published. The video with the associated control signals may be published over a network. The video with the associated control signals may be published to exercise machines and other devices of users as discussed herein. In some embodiments the video may be published separately from the associated control signals. In other embodiments the video with the associated control signals may be one file or more than one file. In some embodiments the associated control signals may be viewable separately from the video and may be represented by text, visuals, or other visual representation. In some embodiments the control signals may be represented in the video by icons, pictures, text, or other visual indicators.

At 2370 the video is displayed at a remote exercise machine and the remote exercise machine is controlled by the associated control signals. A remote exercise machine may be an exercise machine in a home of a user. The video, along with the associated control signals controlling the remote exercise machine, may provide a simulated experience of moving through a virtual environment with a trainer. For example, a video of a trainer running in Atlantis may be displayed on a display of a treadmill controlled by control signals corresponding to a speed of a movement through Atlantis and an incline of a path through Atlantis. This may give a simulated experience to the user of running through Atlantis with the trainer.

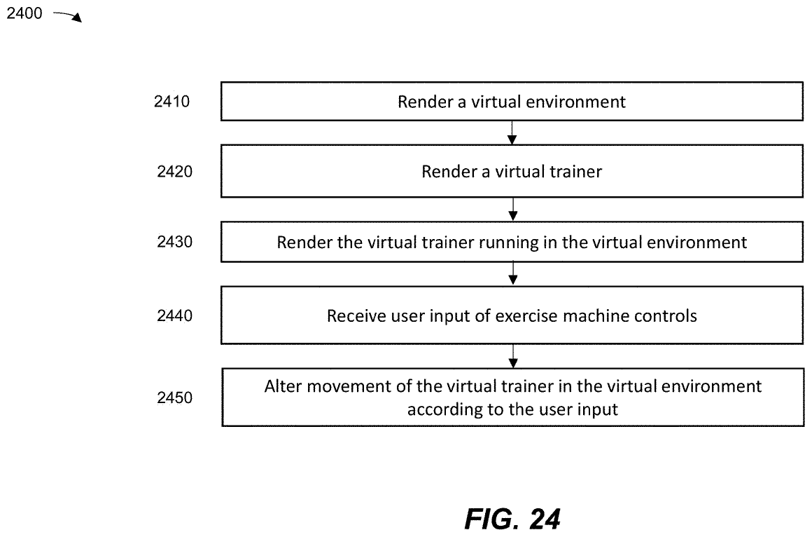

FIG. 24 illustrates a flowchart of an example method 2400 for controlling movement of a virtual trainer through a virtual environment. Additional, fewer, or different operations may be performed in the method, depending on the embodiment. Further, the operations may be performed in the order shown, concurrently, or in a different order. At 2410 a virtual environment is rendered. At 2420 a virtual trainer is rendered. The virtual trainer may be modeled on a real trainer. In some embodiments the virtual trainer may be a character from a movie, TV show, or game. At 2430 the virtual trainer is rendered running in the virtual environment. The virtual trainer may run, cycle, or walk in the virtual environment. The virtual trainer may run in the virtual environment along a predetermined path. In some embodiments the user may choose a direction for the virtual trainer to run. At 2440 user input of exercise machine controls is received at an exercise machine. For example, user input of exercise machine controls for a treadmill includes user input of a speed and incline. At 2450 the movement of the virtual trainer in the virtual environment is altered according to the user input. A speed of the virtual trainer may be altered to match a speed of the user input. For example, a virtual trainer may be running along a path in a virtual environment at 7 miles per hour and transition to running at 5 miles per hour based on a user inputting a speed of 5 miles per hour at a treadmill. In this way, the user can have the experience of running with a trainer customized to their particular needs and ability level. Aspects of this example method may be applied to filming a real-world trainer in a virtual environment. For example, a real-world trainer may be filmed running in a virtual environment and the speed of the trainer may be determined not by pre-defined exercise machine controls, but by real-time input of the trainer or another person. The virtual environment, displayed on the video wall, may be updated so that a speed of movement through the virtual environment matches the speed of the input. The input may be converted into exercise machine controls which are associated with the video of the trainer running in the virtual environment. A remote exercise machine may then display the video and be controlled according to the associated exercise machine controls.

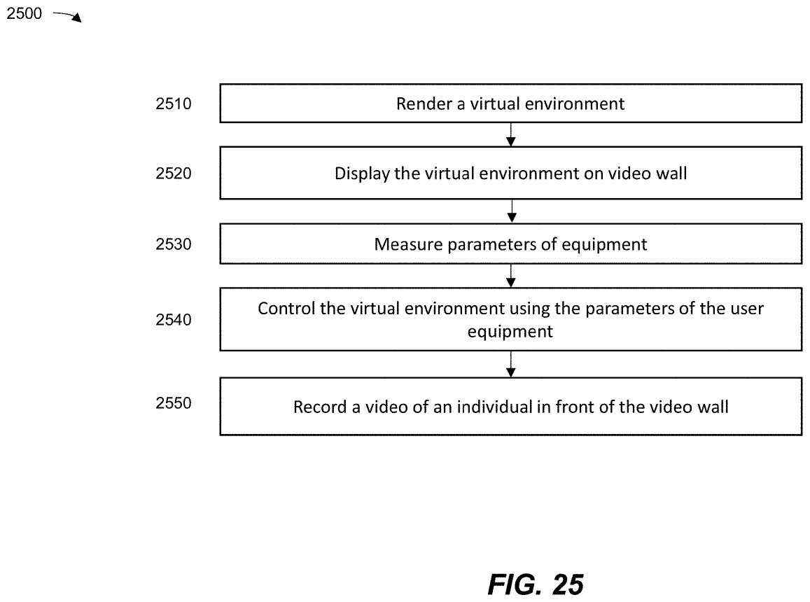

FIG. 25 illustrates a flowchart of an example method 2500 for controlling a virtual environment using equipment. Additional, fewer, or different operations may be performed in the method, depending on the embodiment. Further, the operations may be performed in the order shown, concurrently, or in a different order. At 2510, a processor renders a virtual environment. In some embodiments, the virtual environment is a computer-generated environment. For example, the virtual environment may be a computer-generated environment rendered in UNREAL ENGINE. In other embodiments, the virtual environment may be constructed using photos or video of real-world environments. For example, a series of photos or a video captured while a person is walking down a real-world street may be used to generate a virtual environment incorporating the series of photos or video such that the virtual environment corresponds to the real-world environment. In yet other embodiments, the virtual environment may be a combination of computer-generated and real-world elements.

At 2520, the virtual environment is displayed on a video wall. The video wall may be an LED screen, a series of LED screens, or other type of display.

At 2530, the parameters of equipment are measured. The equipment may be any equipment operated or used by an individual including, but not limited to, a treadmill, a stationary bike, a rower, a stair climber, a wire harness, or other equipment. The parameters of the equipment may include position, orientation, velocity, acceleration, velocity of one or more members of the equipment, resistance, cadence, incline, and other parameters. For example, a position, orientation, incline, and speed of a treadmill may be measured. The parameters may be determined by the individual using the equipment, by another person, or by a computer. In some embodiments, the parameters may be measured using one or more sensors. In other embodiments, the parameters may be measured using a camera. For example, the incline and speed of a treadmill may be measured using sensors or output signals of the treadmill and the orientation of the treadmill may be measured using a camera.

At 2540, the virtual environment is controlled using the parameters of the equipment. The virtual environment may be controlled so as to synchronize the virtual environment with the parameters of the equipment. The virtual environment may be controlled using the parameters of the equipment such that the equipment appears to be in the virtual environment. A perspective of the virtual environment may be such that the equipment appears to be located in the virtual environment. The virtual environment may be controlled using the parameters of the equipment such that the individual using the equipment appears to be in the virtual environment. For example, movement through the virtual environment may be synchronized with a speed of a treadmill such that an individual using the treadmill appears to walk or run through the virtual environment. In another example, movement through the virtual environment may be synchronized with movement of a wire harness such that an individual using the wire harness appears to fly or fall through the virtual environment.

At 2550 a video is recorded of an individual in front of the video wall. The video may appear to show the individual in the virtual environment. The video may be used for various purposes. For example, the video may be an exercise video and the individual may be a trainer using exercise equipment. In another example, the video may be a movie or TV show and the individual may be an actor using a treadmill to realistically appear to walk through the virtual environment. In yet another example, the video may be an instructional video for using the equipment and the individual may be demonstrating use of the equipment in various environments. In yet another example, the video may be a movie or TV show. In yet another example, the individual may be an animal walking on a treadmill.

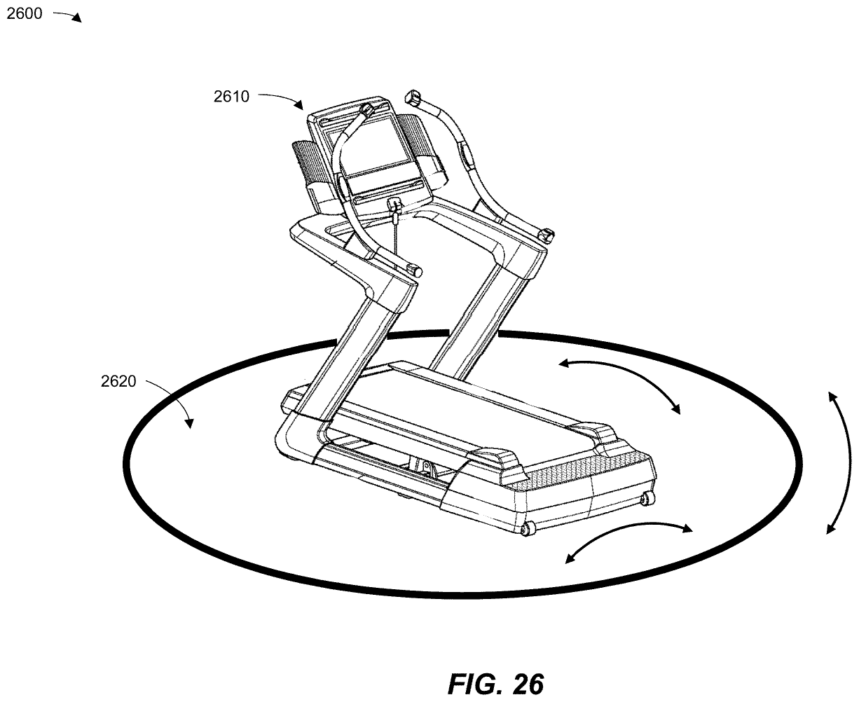

FIG. 26 illustrates an example stage 2620 for equipment 2610 in accordance with one or more embodiments. The stage 2620 may be configured to support the equipment 2610. A treadmill is illustrated as the equipment 2610, but the equipment may be any equipment. The stage 2620 may be configured to rotate about a vertical axis to rotate the equipment 2610. The stage 2620 may be configured to tilt in various directions. The stage 2620 may be configured to tilt about two orthogonal axes in the plane of the stage 2620 such that the equipment 2610 tilts. For example, the stage 2620 may be configured to tilt a treadmill such that a front of the treadmill tilts up and down and the treadmill tilts side to side. The stage 2620 may include an electric motor configured to rotate the stage 2620. The stage 2620 may include hydraulics and/or pneumatics configured to tilt the stage 2620.

The stage 2620 may be used in conjunction with other embodiments disclosed herein. For example, the stage 2620 may be used to support equipment 2610 in the method 2500 of FIG. 25. A rotation and tilt of the stage 2620 may determine a rotation and tilt of the equipment 2610 such that the rotation and tilt of the stage 2620 or the rotation and tilt of the equipment 2610 are used to control the virtual environment displayed on the video wall such that the virtual environment is synchronized with the movement of the equipment such that the individual using the equipment appears to be in the virtual environment. For example, an individual may use a stationary bike on the stage 2620 and the virtual environment may be synchronized with a cadence of pedaling of the individual, a resistance of the stationary bike, and an incline and rotation of the bike and stage 2620.

### INDUSTRIAL APPLICABILITY

Various modifications to the embodiments illustrated in the drawings will now be disclosed.

In general, some example methods disclosed herein may consider mental health, together with physical health, of a person in delivering and/or recommending video programs, such as video workout programs and/or video mental health programs, to users. The delivery and/or recommendation of video programs to the users may effect a positive change in the mental health of the user. In some embodiments, mental health improvement sessions may be provided to users before, after, or combined with workouts to leverage the effectiveness of exercise in treating mental health maladies.

Mental health may be considered by asking the user questions related to their mental health or in other manners. As previously indicated, the questions may specifically relate to a mental health history of the user, a mental health history of the user's family, sleep habits, sleep changes, mood, mood changes, anxiety, depression, stress, confusion, self-esteem, apathy, suicidal thoughts, or other aspects of mental health of the user. For example, users may be asked questions such as “When was the last time that you laughed?”, “Have you lost interest in things you used to enjoy?”, “In the past two weeks, how often have you felt down, depressed, or hopeless?”, “Have you had any thoughts of suicide?”, “How is your sleep?”, “How is your energy?”, “Do you prefer to stay at home rather than going out and doing new things?”, “Are you a worrier?”, “Have you been worrying about simple things you shouldn't be worrying about?”, “Over the past few months of worrying, have you noticed that you have been jittery or on edge?”.

In some embodiments, and based on the answers to such questions or other psychological and/or biological parameters of the user, a mental state of the user may be determined and/or the user may be assisted in determining their mental state. An aspect of a video program may be controlled to effect a positive change in the user's mental health or otherwise influence the mental state of the user and/or one or more existing video programs may be recommended to the user to influence the user's mental state. Alternatively or additionally, one or more custom video programs may be generated for the user on the fly, e.g., by AI/ML. For example, one or more particular workouts, mental health improvement sessions, or other activities that may be performed on or with one or more of the wellness devices herein may be known or suspected to positively influence the mental state of users in a given mental state and the AI/ML may generate a video program that includes the particular workout, mental health improvement session, or other activity. The AI/ML may include in the video program one or more pre-recorded segments or branches of video of an instructor or may generate one or more segments or branches of video of an instructor to include in the video program on the fly, e.g., using a game engine. Alternatively or additionally, the AI/ML may generate and include in the generated segments or branches of video a deepfake depiction of an instructor to guide or direct the user through the particular workout, mental health improvement session, and/or other activity.

Video programs with an instructor guiding or directing users through workouts and/or mental health improvement sessions may include images or video of the instructor and/or may include other images or video from which the instructor is absent. For example, the images or video of a video program executed at a sleep assistance device may include images or video of a night sky or nature scene or other imagery without any images or video of the instructor. In some embodiments, even when a video program lacks images or video of the instructor, the video program may include audio of the instructor guiding or directing users through workouts and/or mental health improvement sessions.

The immersive mental health device 600 of FIGS. 6A and 6B may have a variety of form factors. In FIGS. 6A and 6B, the immersive mental health device 600 includes the housing 604 that is movable between open and closed positions. In other embodiments, the housing 604 may have a clamshell design with a left shell and a right shell that are rotatably joined together, e.g., with a hinge behind the adjustable chair 602, and that may be opened/closed by rotating relative to each other and the adjustable chair 602. In other embodiments, the housing 604 may be large enough to completely enclose the adjustable chair 602 and the user 608 when closed. Alternatively, the adjustable chair 602 may be placed in a room that may serve the same or similar purpose(s) as the housing 604 and the housing 604 may be omitted altogether.

In some embodiments, one or more of the wellness devices herein may connect to a fitness platform, a wellness platform, or a social media platform. For example, one or more of the wellness devices may connect to Icon Health & Fitness's IFIT, which is an Internet connected and interactive fitness platform. Any of the devices, systems, or servers that perform any of the methods disclosed herein or actions thereof may collect parameters of users from such platforms.

Sleep can have a significant impact on mental health. The use of smartphones and other personal electronic devices in bedrooms and/or leading up to bedtime can negatively impact sleep. In some embodiments, sleep assistance devices and/or sleep assistance sessions described herein may encourage users to leave their smartphones or other personal electronic devices outside the users' bedrooms or may include compartments or chambers that hide the smartphones from view to lessen the possibility of the smartphones distracting the users. For example, sleep assistance devices as described herein may include a chamber, e.g., formed or supported in a main body of the sleep assistance device, within which a smartphone may be placed out of view of a user. In some embodiments, the chamber may be a cleaning chamber configured to sanitize the smartphone or other articles placed in the cleaning chamber. For example, the chamber may be configured to sanitize the smartphone or other articles placed therein by emitting ultraviolet (UV) light at the smartphone or other articles. Alternatively or additionally, sleep assistance devices as described herein may include a charge dock, e.g., formed or supported in a main body of the sleep assistance device. The charge dock may include a charger configured to charge a smartphone or other personal electronic device(s) of a user. The charger may include an inductive charger. In some embodiments, the charge dock may be positioned or otherwise configured in the sleep assistance device to hide the smartphone or other personal electronic device(s) from view.

Notwithstanding the negative effect the use of smartphones or other personal electronic devices can have on sleep, some users may desire to remain “connected” at bedtime and/or at night. Accordingly, sleep assistance devices as described herein may be configured to pair with smartphones or other personal electronic devices. In some embodiments, notifications or content on the smartphones or personal electronic devices may be output to the user, e.g., via the display device and/or audio speaker of the sleep assistance device. Alternatively or additionally, the user interface of the sleep assistance device may be used to operate the smartphone or other personal electronic device.

The sleep assistance devices 700a-700e of FIGS. 7A-7H have a variety of form factors and may take other form factors in other embodiments. The sleep assistance devices 700a and 700e are relatively compact devices that may be used on a nightstand or other location while the sleep assistance devices 700b-700d are bulkier devices that may be mounted on or near the user's bed. In other embodiments, sleep assistance devices of any size (e.g., large or small) may be placed or mounted on nightstands, walls, headboards, footboards, ceilings, or other structures and may have any form factors. Examples of other sleep assistance device form factors include nightstands, lamp tables, lamps, smart beds, wall stands, home goods (e.g., vases or other home decorations), or the like.

As previously indicated, some embodiments herein may utilize concepts of accountability, assessment, and/or progress to aid wellness device users and/or other persons to improve their physical and/or mental health. According to some methods herein that make a first person accountable in changing a behavior or mental state, responsive to determining that the first person is vulnerable to relapse the first person may be contacted indirectly by arranging for a second person to directly contact the first person to offer support to the person to avoid relapse. In some embodiments, it may be determined, e.g., by a computing device such as a health care device or server as described herein, that the second person fails to directly contact the first person within a predetermined amount of time from arranging for the second person to directly contact the first person and the method may further include arranging for a third person to directly contact the first person. For example, an app or application on a smartphone or other personal electronic device of the first person or the second person may determine whether the predetermined amount of time has passed since arranging for the second person to directly contact the first person and may notify the computing device that the second person has failed to directly contact the first person with the predetermined amount of time. In response, the computing device may arrange for a third person to directly contact the first person to offer support.

In some embodiments, prior to indirectly contacting the first person, the computing device may request input from the person to select a subset of multiple contacts of the first person to contact the first person when it is determined that the first person is vulnerable to relapse and may receive a selection by the first person of the subset, the subset including the second person. The second person may include a significant other (e.g., spouse, boyfriend, girlfriend), a parent, a sibling, a child, a relative, a friend, a coach, a mentor, a sponsor, or a social media connection.

Arranging for the second person to contact the first person may include sending the second person an e-mail, text message, voice call, voice message, video call, video message, or other communication instructing or asking the second person to contact the first person to offer support. In some embodiments, the second person may be instructed how to offer support to the first person, which may include, e.g., presenting a tutorial video or audio to the second person describing one or more questions to ask the first person or one or more encouraging statements to make to the first person.

In some embodiments, the first person may be contacted to offer support only in response to determining that the person is vulnerable to relapse. In some embodiments, the first person may be contacted at any time to offer support whether or not the first person is vulnerable to relapse. For example, the first person may be contacted periodically or according to a predetermined schedule to offer general support to the first person.

In accordance with common practice, the various features illustrated in the drawings may not be drawn to scale. The illustrations presented in the present disclosure are not meant to be actual views of any particular apparatus (e.g., device, system, etc.) or method, but are merely example representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, it is understood that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the summary, detailed description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

The foregoing description, for purpose of explanation, has been described with reference to specific embodiments. However, the illustrative discussions above are not intended to be exhaustive or to limit the invention as claimed to the precise forms disclosed. Many modifications and variations are possible in view of the above teachings. The embodiments were chosen and described to explain practical applications, to thereby enable others skilled in the art to utilize the invention as claimed and various embodiments with various modifications as may be suited to the particular use contemplated.

A. A method to influence mental state of a user of a wellness device with a video program, the method comprising:

executing, at the wellness device, the video program, the wellness device including one or more moveable members;

continually controlling the one or more moveable members of the wellness device according to the video program;

collecting biological parameters of the user;

collecting psychological parameters of the user;

controlling an aspect of at least one of the video program or an environment of the user based on both the biological parameters and the psychological parameters to influence the mental state of the user.

B. The method of section A, wherein the collecting of the psychological parameters of the user comprises collecting responses of the user to questions relating to mental health of the user.
C. The method of section B or a, further comprising presenting the questions relating to the mental health of the user to the user.
D. The method of section C, wherein the presenting of the questions comprises presenting questions related to at least one of: mental health history of the user, mental health history of the user's family, sleep habits, sleep changes, mood, mood changes, anxiety, depression, stress, confusion, self-esteem, apathy, or suicidal thoughts.
E. The method of one of sections A-D, wherein the collecting occurs at least one of:

before the video program begins;

at a beginning of the video program; or

during the video program.

F. The method of one of sections A-E, wherein:

the video program is a current video program; and

the method further comprises recommending, based on both the biological parameters and the psychological parameters, another video program to the user.

G. The method of section F, wherein:

the video program comprises a first video workout program or video mental health program; and

the other video program comprises a second video workout program or video mental health program.

H. The method of one of sections A-G, wherein:

the collecting occurs before the video program begins; and

the method further comprises recommending, based on both the biological parameters and the psychological parameters, the video program to the user.

I. The method of one of sections A-H, wherein:

the video program includes a video that is continuously displayed to the user during the video program; and

the controlling of the aspect comprises controlling content of the video of the video program.

J. The method of one of sections A-I, wherein the controlling of the aspect comprises controlling an output of a sun lamp in the environment of the user.
K. The method of one of sections A-J, wherein the controlling of the aspect comprises controlling both a first aspect of the video program and a second aspect of the environment of the user in coordination.
L. The method of one of sections A-K, wherein the collecting of the biological parameters comprises recording brain waves of the user.
M. The method of one of sections A-L, wherein the executing the video program at the wellness device includes guiding the user through a mental health improvement session.
N. The method of section M, wherein the mental health improvement session comprises at least one of: a mindfulness session, a breathing session, a yoga session, a sleep assistance session, or a therapy session.
O. The method of section M, wherein the executing the video program at the wellness device further includes guiding the user through a workout.
P. The method of one of sections A-O, further comprising determining, by at least one of an artificial intelligence or machine learning, the aspect to be controlled based on both the biological parameters and the psychological parameters.
Q. The method of one of sections A-P, wherein the wellness devices comprises an exercise machine.
R. The method of one of sections A-Q, wherein:

the wellness device comprises at least one of: an adjustable chair, a haptic device, a display device, a scent dispenser, a heater element, a cooler element, a compression member, a humidity control element, a speaker, a fan, or a light; and

the controlling of the aspect comprises controlling at least one of:
recline or tilt of the adjustable chair;
vibrational movement of the haptic device;
applied compression by the compression member;
dispensing of scent from the scent dispenser;
at least one of ambient temperature, humidity, or airflow via at least one of the heater element, the cooler element, the fan, the light, or the humidity control element;
light from at least one of the light or the display device;
audio content from the speaker; or
video content from the display device.
   S. A method to influence mental state of a user of an immersive mental health device with a video mental health program, the method comprising:

executing, at the immersive mental health device, the video mental health program, the immersive mental health device including one or more moveable members;

continually controlling the one or more moveable members of the immersive mental health device according to the video mental health program;

collecting biological parameters of the user;

collecting psychological parameters of the user;

controlling an aspect of at least one of the video mental health program or an environment of the user based on both the biological parameters and the psychological parameters to influence the mental state of the user.

T. A method to influence mental state of a user of an exercise machine with a video workout program, the method comprising:

executing, at the exercise machine, the video workout program, the exercise machine including one or more moveable members;

continually controlling the one or more moveable members of the exercise machine according to the video workout program;

collecting biological parameters of the user;

collecting psychological parameters of the user;

controlling an aspect of at least one of the video workout program or an environment of the user based on both the biological parameters and the psychological parameters to influence the mental state of the user.

U. An immersive mental health device, comprising:

an adjustable chair including one or more movable members configured to adjustably support the user;

a processing unit communicatively coupled to the adjustable chair; and

a non-transitory computer-readable medium communicatively coupled to the processing unit, the non-transitory computer-readable medium having computer-executable instructions stored thereon that are executable by the processing unit to perform or control performance of operations comprising:
executing, at the immersive mental health device, a video mental health program;
continually controlling the adjustable chair according to the video mental health program;
collecting biological parameters of the user;
collecting psychological parameters of the user;
controlling an aspect of at least one of the video mental health program or an environment of the user based on both the biological parameters and the psychological parameters to influence the mental state of the user.
   V. The immersive mental health device of section U, further comprising a housing movably coupled to the adjustable chair, the housing movable between an open position and a closed position, the housing configured to at least partially enclose the user on the adjustable chair when the housing is in the closed position.
   W. The immersive mental health device of sections U or V, further comprising:

an emissive display coupled to an inner surface of the housing and positioned to be in view of the user when the housing is in the closed position; or

a projector positioned to project video content onto the inner surface of the housing at a location in view of the user when the housing is in the closed position.

X. The immersive mental health device of sections V or W, further comprising at least one of a haptic device, a scent dispenser, a heater element, a cooler element, a compression member, a humidity control element, a speaker, a fan, or a light communicatively coupled to the processing unit and positioned to output at least one of haptic feedback, scent, heating, cooling, compression, humidity, audio, airflow, or light to the user when the user is on the adjustable chair and at least partially enclosed by the housing.
Y. A sleep assistance device, comprising:

a main body;

a processing unit supported in the main body;

an audio speaker supported in the main body and communicatively coupled to the processing unit;

a light source supported in the main body and communicatively coupled to the processing unit;

a scent dispenser supported in the main body and communicatively coupled to the processing unit;

a display device supported in the main body and communicatively coupled to the processing unit; and

a non-transitory computer-readable medium supported in the main body and communicatively coupled to the processing unit, the non-transitory computer-readable medium having computer-executable instructions stored thereon that are executable by the processing unit to perform or control performance of operations comprising:
collecting one or more parameters about a user of the sleep assistance device, the one or more parameters including at least one of one or more biological parameters or one or more psychological parameters;
generating a current sleep assistance session based on the one or more parameters, the current sleep assistance session configured to assist the user to reach and remain in a sleep state; and
executing the current sleep assistance session at the sleep assistance device, including coordinating operation of the audio speaker, the light source, the scent dispenser, and the display device to output coordinated aural, visual, and olfactory stimuli to the user that are configured to assist the user to reach and remain in the sleep state.
   Z. The sleep assistance device of section Y, wherein:

the processing unit is communicatively coupled to one or more sensors in operational proximity to the user as the user sleeps; and

the collecting of the one or more parameters includes collecting from the one or more sensors one or more measurements of the user generated as the user sleeps during a prior sleep session of the user.

AA. The sleep assistance device of section Z, wherein the generating of the current sleep assistance session comprises modifying a prior sleep assistance session based on a reaction of the user to the prior sleep assistance session reflected in the one or more measurements of the user generated as the user sleeps during the prior sleep session of the user.
BB. The sleep assistance device one of sections Y-AA, wherein the collecting of the one or more parameters comprises collecting the one or more parameters from an online fitness profile, wellness profile, or social media profile of the user.
CC. The sleep assistance device of section BB, wherein the one or more parameters from the online fitness profile, mental health profile, or social media profile of the user include one or more parameters derived or collected in association with administration of a video workout program or a video mental health program to the user at an exercise machine or an immersive mental health device.
DD. The sleep assistance device of one of sections Y-CC, wherein:

the display device comprises a projector;

the sleep assistance device further comprises a vapor dispenser communicatively coupled to the processing unit and configured to output a vapor sheet; and

the projector is configured to project visual stimuli onto the vapor sheet.

EE. The sleep assistance device of one of sections Y-DD, wherein the light source comprises at least one of:

a wavelength-controllable light source;

a red light source; or

a blue light source.

FF. The sleep assistance device of one of sections Y-EE, wherein the scent dispenser comprises a container and a diffuser communicatively coupled to the container to diffuse liquid scent from the container into an environment of the sleep assistance device.
GG. The sleep assistance device of section FF, wherein the container comprises at least one of a refillable scent cartridge, a disposable scent cartridge, or a biodegradable scent cartridge.
HH. The sleep assistance device of one of sections Y-GG, wherein:

the sleep assistance device further comprises a cleaning chamber supported in the main body and communicatively coupled to the processing unit; and

the cleaning chamber is configured to sanitize an article placed in the cleaning chamber.

II. The sleep assistance device of section HH, wherein the cleaning chamber is configured to sanitize the article placed in the cleaning chamber by emitting ultraviolet (UV) light at the article.
JJ. The sleep assistance device of one of sections Y-II, further comprising a charge dock formed in the main body, the charge dock including a charger configured to charge a personal electronic device.
KK. The sleep assistance device of section JJ, wherein the charger comprises an inductive charger.
LL. The sleep assistance device of section JJ or KK, wherein the charge dock is configured to hide the personal electronic device from view.
MM. The sleep assistance device of one of sections Y-LL, the operations further comprising:

generating a current wake assistance session at the sleep assistance device based on the one or more parameters, the current wake assistance session configured to assist the user to awake from the sleep state; and

executing the current wake assistance session at the sleep assistance device, including coordinating operation of the audio speaker, the light source, the scent dispenser, and the display device to output coordinated aural, visual, and olfactory stimuli to the user that are configured to wake the user from the sleep state.

NN. The sleep assistance device of section MM, wherein executing the current wake assistance session comprises operating at least one of the light source or the display device to output time-varying light that mimics time-varying light from a sunrise.
OO. A smart blanket, comprising:

a blanket, including:
a bottom layer;
a top layer; and
a temperature control layer positioned between the bottom layer and the top layer;

one or more sensors coupled to the blanket;

a haptic device coupled to the blanket; and

a control device coupled to the blanket, the control device including a processing unit communicatively coupled to the temperature control layer, the one or more sensors, and the haptic device and a non-transitory computer-readable medium communicatively coupled to the processing unit, the non-transitory computer-readable medium having computer-executable instructions stored thereon that are executable by the processing unit to perform or control performance of operations comprising:
collecting, by the one or more sensors, one or more parameters about a user of the smart blanket; and
operating the temperature control layer and the haptic device based on the one or more parameters to assist the user to reach and remain in a sleep state.
   PP. The smart blanket of section OO, wherein the temperature control layer comprises:

a heater sublayer; and

a cooler sublayer.

QQ The smart blanket of section PP, wherein the heater sublayer comprises electrical heating wires.
RR. The smart blanket of section PP or QQ, wherein the cooler sublayer comprises one or more coolant conduits coupled to a coolant source.
SS The smart blanket of one of sections OO-RR, wherein:

the temperature control layer comprises a conduit with vents through the bottom layer; and

the smart blanket further comprises a control box with a fan and a heater element and a hose coupled between the control box and the temperature control layer, the heater element configured to generate heated air, the fan configured to circulate the heated air or air at room temperature through the temperature control layer and out the vents.

TT. The smart blanket of one of sections OO-SS, wherein the one or more sensors include at least one of a heart rate sensor, a body temperature sensor, a motion sensor, a respiratory sensor, or a microphone.
UU. The smart blanket of one of sections OO-TT, wherein:

the collecting of the one or more parameters about the user includes measuring a breathing rate of the user and a body temperature of the user; and

the operating of the temperature control layer and the haptic device includes:
operating the temperature control layer based on the body temperature of the user to adjust the body temperature of the user towards a target body temperature; and
operating the haptic device based on the breathing rate of the user to adjust the breathing rate of the user towards a target breathing rate.
   VV. A smart yoga mat system, comprising

a yoga mat;

a plurality of lights distributed throughout the yoga mat; and

an electronics unit coupled to the yoga mat, the electronics unit including:
a processing unit;
an audio speaker supported in the electronics unit and communicatively coupled to the processing unit;
a display device supported in the electronics unit and communicatively coupled to the processing unit;
a scent dispenser supported in the electronics unit and communicatively coupled to the processing unit; and
a non-transitory computer-readable medium supported in the electronics unit and communicatively coupled to the processing unit;

wherein the non-transitory computer-readable medium has computer-executable instructions stored thereon that are executable by the processing unit to perform or control performance of operations comprising:
guiding the user through a yoga session with aural, visual, and olfactory stimuli output by the electronics unit;
selectively lighting subsets of the plurality of lights to identify proper placement of one or more appendages of the user on the yoga mat for a given pose.
   WW. The smart yoga mat system of section VV, wherein:

the operations further comprise monitoring poses of the user during performance of the yoga session by the user; and

the selectively lighting of the subsets of the plurality of lights occurs in response to determining during the monitoring of the poses that a placement by the user of at least one appendage of the user for the given pose is incorrect.

XX. The smart yoga mat system of section VV or WW, further comprising a camera operatively coupled to the processing unit and configured to capture at least one of an image or video of the user performing a yoga pose.
YY. The smart yoga mat system of section XX, wherein the operations further comprise:

displaying, by the display device, the image or video of the user performing the yoga pose to the user; and

providing instructions, by at least one of the display device, the audio speaker, or the plurality of lights, to the user to adjust the yoga pose to match a target yoga pose.

ZZ. A method to make a person accountable to change behavior or mental state from an initial behavior or mental state to a target behavior or mental state, the method comprising:

collecting, by one or more sensors in operational proximity to the person, one or more parameters about the person;

determining based on the one or more parameters that the person is vulnerable to relapse to the initial behavior or mental state; and

responsive to determining that the person is vulnerable to relapse to the initial behavior or mental state, contacting the person to offer support to the person to avoid relapse.

AAA. The method of section ZZ, wherein the contacting of the person comprises directly contacting the person via e-mail, text message, voice call, voice message, video call, or video message.
BBB. The method of section AAA, wherein the e-mail, text message, voice call, voice message, video call, or video message includes a computer-generated deepfake representation of someone known to the person.
CCC. The method of one of sections ZZ-BBB, wherein:

the person is a first person; and

the contacting of the first person comprises indirectly contacting the first person by arranging for a second person to directly contact the first person.

DDD. The method of section CCC, further comprising:

determining that the second person fails to directly contact the first person within a predetermined amount of time from arranging for the second person to directly contact the first person; and

arranging for a third person to directly contact the first person.

EEE. The method of one of section CCC or DDD, further comprising, prior to indirectly contacting the first person:

requesting input from the person to select a subset of multiple contacts of the first person to contact the first person when it is determined that the first person is vulnerable to relapse; and

receiving a selection by the first person of the subset, the subset including the second person.

FFF. The method of one of sections CCC-EEE, wherein the second person comprises a significant other, a parent, a sibling, a child, a relative, a friend, a coach, a mentor, a sponsor, or a social media connection.
GGG. The method of one of sections CCC-FFF, further comprising instructing the second person how to offer support to the first person.
HHH. The method of section GGG, wherein the instructing includes presenting a tutorial video or audio to the second person describing one or more questions to ask the first person or one or more encouraging statements to make to the first person.
III. The method of one of sections ZZ-HHH, wherein the initial behavior or mental state comprises at least one of:

smoking;

vaping;

being sedentary;

overconsuming food;

consuming or overconsuming alcohol;

consuming or overconsuming drugs;

insomnia;

anxiety; or

depression.

JJJ. The method of one of sections ZZZ-III, wherein the target behavior or mental state comprises at least one of:

not smoking;

not vaping;

exercising;

not overconsuming food;

not consuming or overconsuming alcohol;

not consuming or overconsuming drugs;

sleeping; or

mental equilibrium.

KKK. The method of one of sections ZZ-JJJ, wherein the collecting, by the one or more sensors, the one or more parameters about the person comprises at least one of:

collecting a parameter by a sensor of a personal electronic device borne by the person;

collecting a parameter by natural language processing of a journal entry of the person entered into a digital journal by the person;

collecting a parameter by an exercise machine used by the person to perform a workout or a mental health improvement session;

collecting a parameter by a digital device used by the person to play a game;

collecting a parameter by an immersive mental health device used by the person to perform a mental health improvement session; or

collecting a parameter from a fitness profile, a wellness profile, or a social medial profile of the person.

LLL. The method of one of sections ZZ-KKK, further comprising:

determining that the person relapsed to the initial behavior;

determining a relapse time at which the person relapsed to the initial behavior; and

analyzing one or more previous parameters of the person captured during an interval of a predetermined duration that begins prior to the relapse time and terminates at the relapse time to identify one or more indications in the one or more previous parameters of the person that the person was vulnerable to the relapse.

MMM. The method of section LLL, wherein the determining based on the one or more parameters that the user is vulnerable to relapse to the initial behavior comprises identifying one or more current indications in the one or more parameters that are similar or identical to the one or more indications identified in the one or more previous parameters.
NNN. The method of one of sections ZZ-MMM, further comprising contacting the person periodically or according to a predetermined schedule to offer general support to the person.
OOO. A method to improve a mental health of a user of one or more wellness devices, the method comprising:

executing, at the one or more wellness devices of the user, a plurality of video programs over time for the user, the plurality of video programs configured to influence a mental state of the user;

monitoring one or more parameters of the user over time, the one or more parameters including a first parameter;

plotting the first parameter of the user as a function of time; and

presenting a plot of the first parameter as a function of time to the user as an indication of an effect of the plurality of video programs on the user.

PPP. The method of section OOO, wherein the monitoring of the one or more parameters of the user over time includes collecting at least one of passive feedback from the user or active feedback from the user.
QQQ. The method of section PPP, wherein the passive feedback includes at least one of:

heart rate;

respiratory rate;

palm perspiration amount;

pupil dilation amount;

pupil dilation speed; or sleep duration.

RRR. The method of section PPP or QQQ, wherein the active feedback includes at least one of a user response to a question related to mental health of the user or a journal entry of the user entered into a digital journal by the user.
SSS. The method of one of sections OOO-RRR, wherein the plurality of video programs include at least one of a video workout program and a video mental health program.
TTT. The method of one of sections OOO-SSS, further comprising recommending one or more of the plurality of video programs to the user based on the one or more parameters.
UUU. A method comprising:

rendering, by a processor, a virtual environment;

associating, by the processor, exercise machine control signals with the virtual environment;

displaying, by the processor, the virtual environment on a video wall;

receiving, by the processor, a video of a trainer on an exercise machine in front of the video wall displaying the virtual environment, wherein the exercise machine has one or more parameters which are controlled according to the exercise machine control signals associated with the virtual environment;

associating, by the processor, the control signals with the video of the trainer in the virtual environment; and

publishing the video with the associated control signals for use on a remote exercise machine.

VVV. The method of section UUU wherein associating, by the processor, exercise machine control signals with the virtual environment includes generating checkpoints in the virtual environment at which the control signals are updated.
VVV1. The method of section VVV wherein checkpoints are generated at regular intervals specified by a distance in the virtual environment.
VVV2. The method of section VVV wherein checkpoints are generated at regular intervals specified by a time period during the movement through the virtual environment.
VVV3. The method of section VVV wherein checkpoints are generated at regular intervals based on a period of time required for actuators of the exercise machine to execute the control signals.
WWW. The method of any of sections VVV-VVV3 wherein associating, by the processor, exercise machine control signals with the video of the trainer in the virtual environment includes associating the checkpoints and corresponding control signals with timestamps of the video of the trainer in the virtual environment.
XXX. The method of any of sections UUU-WWW wherein associating, by the processor, exercise machine control signals with the video of the trainer in the virtual environment includes associating the exercise machine control signals with timestamps of the video of the trainer in the virtual environment.
XXX1. The method of any of sections UUU-XXX wherein the one or more parameters of the exercise machine are controlled by one or more actuators of the exercise machine.
XXX2. The method of section XXX1 wherein the one or more actuators of the exercise machine include at least one of an actuator to control the speed of an endless belt, an actuator to control the incline of an endless belt, an actuator to control the resistance on a flywheel, and an actuator to control the incline of an exercise machine.
XXX3. The method of any of sections UUU-XXX2 wherein displaying the virtual environment on a video wall includes tracking the motion of a camera and altering the display of the virtual environment on the video such that the view of the virtual environment displayed on the video wall corresponds to the movement of the camera so as to maintain a perspective of the camera so as to create the illusion of the camera being in the virtual environment.
YYY. The method of any of sections UUU-XXX3 wherein associating, by the processor, exercise machine control signals with the video of the trainer in the virtual environment comprises:

rendering, by the processor, the virtual environment;

recording, by the processor, movement through the virtual environment;

measuring, by the processor, a speed of the movement through the virtual environment and the incline of the virtual environment along a path of the movement through the virtual environment;

converting, by the processor, the speed of the movement through the virtual environment and the incline of the virtual environment along a path of the movement through the virtual environment into control signals configured to adjust parameters of an exercise machine, wherein the control signals are modified by a scaling factor; and

associating, by the processor, the control signals with corresponding points in the virtual environment.

ZZZ. The method of section YYY wherein associating, by the processor, the control signals with corresponding points in the virtual environment includes associating the control signals with timestamps of the video of the trainer in the virtual environment.
AAAA. A method comprising:

rendering, by a processor, a virtual environment;

displaying, by the processor, the virtual environment on a video wall;

receiving, by the processor, a video of a trainer on an exercise machine in front of the video wall displaying the virtual environment, wherein the virtual environment is controlled by input corresponding to control signals of the exercise machine;

associating, by the processor, the control signals corresponding to the input with the video of the trainer in the virtual environment; and

publishing the video with the associated control signals for use on a remote exercise machine.

BBBB. A method comprising:

rendering, by a processor, a virtual environment;

associating, by the processor, exercise machine control signals with the virtual environment;

displaying, by the processor, the virtual environment on a video wall;

updating, by the processor, the display of the virtual environment according to the exercise machine control signals associated with the virtual environment;

receiving, by the processor, a video of a trainer on an exercise machine in front of the video wall displaying the virtual environment,
wherein the exercise machine has one or more parameters which are controlled according to the exercise machine control signals associated with the virtual environment, and
wherein the one or more parameters are synchronized with the updating of the display of the virtual environment so as to simulate movement of the trainer through the virtual environment;

associating, by the processor, the control signals with the video of the trainer in the virtual environment; and

publishing the video with the associated control signals for use on a remote exercise machine.

CCCC. A method comprising:

rendering, by a processor of an exercise machine, a virtual environment;

rendering, by the processor, a virtual trainer moving through the virtual environment;

displaying, by the processor, the movement of the virtual trainer through the virtual environment;

receiving, by the processor, user input corresponding to control signals for controlling one or more parameters of the exercise machine; and

updating, by the processor, the movement of the virtual trainer through the virtual environment according to the user input.

DDDD. A method comprising:

rendering, by a processor of an exercise machine, a virtual environment;

displaying, by a processor of the exercise machine, the virtual environment

receiving, by the processor, user input corresponding to control signals for controlling one or more parameters of the exercise machine; and

updating, by the processor, the display of the virtual environment according to the user input so as to simulate movement of the user through the virtual environment.

EEEE. The method of section DDDD wherein the virtual environment includes one or more figures, wherein each figure represents a progress of another user through the virtual environment.
FFFF. A method comprising:

rendering, by a processor, a virtual environment;

displaying, by the processor, the virtual environment on a video wall;

updating, by the processor, the display of the virtual environment according to parameters of equipment, wherein the display of the virtual environment is updated such that an individual using the equipment appears to be located in the virtual environment; and

receiving, by the processor, a video of the individual using the equipment in front of the video wall displaying the virtual environment.

GGGG. The method of section FFFF, wherein the display of the virtual environment is synchronized with the parameters of the equipment so as to simulate movement of the individual through the virtual environment.
HHHH. The method of any of sections FFFF or GGGG, wherein the equipment is a treadmill.
IIII. The method of any of sections FFFF-HHHH, wherein the individual is an actor.
JJJJ. The method of any of sections FFFF-IIII, wherein the virtual environment is based on a real-world location.
KKKK. The method of any of sections FFFF-JJJJ, wherein the individual controls the parameters of the equipment.
LLLL. The method of any of sections FFFF-KKKK, further comprising:

providing a stage upon which the equipment rests, wherein the stage is configured to rotate in two directions and tilt along two axes in order to rotate and tilt the equipment.

MMMM. The method of LLLL, wherein the display of the virtual environment is synchronized with the movement of the stage such that the individual using the equipment appears to be located in the virtual environment.
NNNN. A system comprising:

a processor configured to:
render a virtual environment;
display the virtual environment on a video wall;
update the display of the virtual environment according to parameters of equipment, wherein the display of the virtual environment is updated such that an individual using the equipment appears to be located in the virtual environment; and
receive a video of the individual using the equipment in front of the video wall displaying the virtual environment.
   OOOO. The system of section NNNN, wherein the processor is configured to synchronize the display of the virtual environment with the parameters of the equipment so as to simulate movement of the individual through the virtual environment.
   PPPP. The system of sections NNNN or OOOO wherein the equipment is a treadmill.
   QQQQ. The system of any of sections NNNN-PPPP wherein the individual is an actor.
   RRRR. The system of any of sections NNNN-QQQQ wherein the virtual environment is based on a real-world location.
   SSSS. The system of any of sections NNNN-RRRR wherein the parameters of the equipment are controlled by the individual.
   TTTT. The system of any of sections NNNN-SSSS further comprising a stage, wherein the stage is configured to rotate in two directions and tilt along two axes in order to rotate and tilt the equipment.
   UUUU. The system of any of sections NNNN-TTTT wherein the processor is configured to synchronize the display of the virtual environment with the movement of the stage such that the individual using the equipment appears to be located in the virtual environment.

1. A method comprising:
rendering, by a processor, a virtual environment;
associating, by the processor, exercise machine control signals with the virtual environment;
displaying, by the processor, the virtual environment on a video wall;
receiving, by the processor, a video of a trainer on an exercise machine in front of the video wall displaying the virtual environment, wherein the exercise machine has one or more parameters which are controlled according to the exercise machine control signals associated with the virtual environment;
associating, by the processor, the control signals with the video of the trainer in the virtual environment; and
publishing the video with the associated control signals for use on a remote exercise machine.

2. The method of claim 1 wherein associating, by the processor, exercise machine control signals with the virtual environment includes generating checkpoints in the virtual environment at which the control signals are updated.

3. The method of claim 2 wherein checkpoints are generated at regular intervals specified by a distance in the virtual environment.

4. The method of claim 2 wherein checkpoints are generated at regular time intervals.

5. The method of claim 2 wherein associating, by the processor, exercise machine control signals with the video of the trainer in the virtual environment includes associating the checkpoints and corresponding control signals with timestamps of the video of the trainer in the virtual environment.

6. The method of claim 1 wherein associating, by the processor, exercise machine control signals with the video of the trainer in the virtual environment includes associating the exercise machine control signals with timestamps of the video of the trainer in the virtual environment.

7. The method of claim 1 wherein the one or more parameters of the exercise machine are controlled by one or more actuators of the exercise machine, wherein the one or more actuators of the exercise machine include at least one of an actuator to control the speed of an endless belt, an actuator to control the incline of an endless belt, an actuator to control the resistance on a flywheel, and an actuator to control the incline of an exercise machine.

8. A system comprising:
a processor configured to:
render a virtual environment;
receive exercise machine control signals;
associate the exercise machine control signals with the virtual environment;
receive a video of a trainer on an exercise machine in front of a video wall displaying the virtual environment, wherein the exercise machine has one or more parameters which are controlled according to the exercise machine control signals associated with the virtual environment;
associate the control signals with the video of the trainer in the virtual environment; and
publish the video with the associated control signals for use on a remote exercise machine.

9. The system of claim 8 wherein associating exercise machine control signals with the virtual environment includes generating checkpoints in the virtual environment at which the control signals are updated.

10. The system of claim 9 wherein generating checkpoints in the virtual environment includes generating checkpoints at regular intervals specified by a distance in the virtual environment.

11. The system of claim 9 wherein generating checkpoints in the virtual environment includes generating checkpoints at regular time intervals.

12. The system of claim 9 wherein associating exercise machine control signals with the video of the trainer in the virtual environment includes associating the checkpoints and corresponding control signals with timestamps of the video of the trainer in the virtual environment.

13. The system of claim 8 wherein associating exercise machine control signals with the video of the trainer in the virtual environment includes associating the exercise machine control signals with timestamps of the video of the trainer in the virtual environment.

14. The system of claim 8 wherein the one or more parameters of the exercise machine are controlled by one or more actuators of the exercise machine, wherein the one or more actuators of the exercise machine include at least one of an actuator to control the speed of an endless belt, an actuator to control the incline of an endless belt, an actuator to control the resistance on a flywheel, and an actuator to control the incline of an exercise machine.

15. A non-transitory computer medium including instructions which, when executed by a processor, cause the processor to:
render a virtual environment;
associate exercise machine control signals with the virtual environment;
receive a video of a trainer on an exercise machine in front of a video wall displaying the virtual environment, wherein the exercise machine has one or more parameters which are controlled according to the exercise machine control signals associated with the virtual environment;
associate the control signals with the video of the trainer in the virtual environment; and
publish the video with the associated control signals for use on a remote exercise machine.

16. The non-transitory computer medium of claim 15 wherein associating exercise machine control signals with the virtual environment includes generating checkpoints in the virtual environment at which the control signals are updated.

17. The non-transitory computer medium of claim 16 wherein generating checkpoints in the virtual environment includes generating checkpoints at regular intervals specified by a distance in the virtual environment.

18. The non-transitory computer medium of claim 16 wherein generating checkpoints in the virtual environment includes generating checkpoints at regular time intervals.

19. The non-transitory computer medium of claim 16 wherein associating exercise machine control signals with the video of the trainer in the virtual environment includes associating the checkpoints and corresponding control signals with timestamps of the video of the trainer in the virtual environment.

20. The non-transitory computer medium of claim 15 wherein associating exercise machine control signals with the video of the trainer in the virtual environment includes associating the exercise machine control signals with timestamps of the video of the trainer in the virtual environment.
